(19) United States
(12) Patent Application Publication
(10) Pub. No.: US 2025/0144243 A1
(43) Pub. Date: May 8, 2025

(54) AAV CAPSIDS FOR IMPROVED HEART TRANSDUCTION AND DETARGETING OF LIVER

(71) Applicant: The Trustees of the University of Pennsylvania, Philadelphia, PA (US)

(72) Inventors: Jacob Hoffman, Philadelphia, PA (US); Joshua Joyner Sims, Villanova, PA (US)

(73) Assignee: The Trustees of the University of Pennsylvania, Philadelphia, PA (US)

(21) Appl. No.: 18/730,384

(22) PCT Filed: Jan. 24, 2023
(86) PCT No.: PCT/US2023/061167

§ 371 Date: Jul. 19, 2024

Related U.S. Application Data
(60) Provisional application No. 63/386,572, filed on Dec. 8, 2022.
(60) Provisional application No. 63/375,005, filed on Sep. 8, 2022.
(60) Provisional application No. 63/302,912, filed on Jan. 25, 2022.

(51) Int. Cl. A61K 48/00; A61K 38/17; C12N 15/86

(52) U.S. Cl. CPC A61K 48/0058; A61K 38/1719; C12N 15/86; C12N 2750/14122; C12N 2750/14143; C12N 2750/14152

(57) ABSTRACT

Provided herein are novel AAV capsids and recombinant AAV vectors comprising the same. In certain embodiment, vectors comprising a novel AAV capsid show improved transduction of cardiac tissue and/or reduced targeting of liver tissue compared to a prior art AAV capsid.

### REFERENCE TO AN ELECTRONIC SEQUENCE LISTING

The contents of the electronic sequence listing (“21-9720PCT.xml”; Size: 43,658 bytes; and Date of Creation: Jan. 22, 2025) is herein incorporated by reference in its entirety.

### BACKGROUND OF THE INVENTION

Adeno-associated virus (AAV) vectors are safe and effective gene transfer vehicles used for several clinical indications. Recombinant AAV vectors have a vector genome lacking AAV coding sequences packaged in an AAV capsid. The AAV capsid is icosahedral in structure and is comprised of 60 of viral protein (VP) monomers (VP1, VP2, and VP3) in a 1:1:10 ratio (Xie Q, et al. Proc Natl Acad Sci USA. 2002; 99(16):10405-10). The entirety of the VP3 protein sequence (519aa) is contained within the C-terminus of both VP1 and VP2, and the shared VP3 sequences are primarily responsible for the overall capsid structure. Due to the structural flexibility of the VP1/VP2 unique regions and the low representation of VP1 and VP2 monomers relative to VP3 monomers in the assembled capsid, VP3 is the only capsid protein to be resolved via x-ray crystallography (Nam H J, et al. J Virol. 2007; 81(22):12260-71).

The natural tropism of AAV vectors for the liver represents a major impediment to the expansion of AAV gene therapy into clinical indications affecting non-liver peripheral organs such as the heart. Recent deaths and adverse events associated with liver toxicity in high-dose AAV gene therapy trials targeting peripheral organs exemplify this problem.

There remains a need in the art for AAV vectors with improved targeting of select cell and tissue types, including vectors that detarget the liver while retaining the ability to transduce peripheral organs.

### SUMMARY OF THE INVENTION

In one aspect, provided herein is a recombinant adeno-associated virus (rAAV) having an AAV clade F capsid comprising a mutant galactose binding site, wherein the mutant galactose binding site comprises (a) any amino acid residue (X) at position 446 (Y446X), (b) an arginine at position 470 (N470R), and (c) a histidine at position 503 (W503H) when residue positions are determined using the residue numbers of SEQ ID NO: 10 as a reference, and a vector genome comprising a 5′ inverted terminal repeat (ITR) sequence, a coding sequence for a gene product, regulatory sequences operably linked to the coding sequence that direct expression of the gene product, and a 3′ ITR sequence. In certain embodiments, the mutant capsid comprises: (a) H at position 446 (HRH); (b) S at position 446 (SRH); (c) V at position 446 (VRH); (d) G at position 446 (GRH); (e) F at position 446 (FRH); (f) T at position 446 (TRH); or (g) S at position 446 (SRH). In certain embodiments, the parental AAV clade F capsid is an AAV9, AAVhu68, AAVhu31, AAVhu32, AAVhu95, or AAVhu96 capsid, or a mutant or variant of any of the aforementioned.

In another aspect, provided herein is a method for generating an a rAAV comprising a mutant capsid derived from a parental AAV capsid having liver specificity, the method comprising culturing a packaging host cell comprising: (a) a nucleic acid sequence encoding a mutant AAV Clade F capsid operably linked to regulatory control sequences that direct its expression in the packaging host cell, wherein the encoded capsid protein comprises (i) any amino acid residue (X) at position 446 (Y446X), (ii) an arginine at position 470 (N470R), and (iii) a histidine at position 503 (W503H), where the amino acid residue positions are determined using the residue numbers of SEQ ID NO: 10 as a reference, (b) a nucleic acid molecule comprising a vector genome comprising a 5′ inverted terminal repeat (ITR) sequence, a coding sequence for a gene product, regulatory sequences operably linked to the coding sequence that direct expression of the gene product, and a 3′ ITR sequence; and (c) helper functions necessary for packaging the vector genome of (b) into the mutant clade F capsid. In certain embodiments, the parental AAV capsid is an AAV9, AAVhu68, AAVhu31, AAVhu32, AAVhu95, or AAVhu96 capsid, or a mutant or variant of any of the aforementioned.

In another aspect, provided herein is an rAAV produced according to the methods disclosed.

In another aspect, provided herein is a method for reducing liver toxicity associated with delivery of an rAAV vector, the method comprising delivering to a subject an rAAV disclosed herein.

In a further aspect, provided herein is a method for improved delivery of a gene product to cardiac cells or tissue, the method comprising administering to a subject the rAAV disclosed herein.

In yet another aspect, provided herein is a nucleic acid comprising a sequence encoding a mutant AAV Clade F VP1 protein having a mutant galactose binding pocket which comprises (a) any amino acid residue (X) at position 446 (Y446X), (b) arginine at position 470 (N470R), and (c) histidine at position 503 (W503H), where the amino acid residue positions are determined using the residue numbers of SEQ ID NO: 10 as a reference, the VP1 protein coding sequence being operably linked to expression control sequences which direct its expression in a packaging host cell. In certain embodiments, the nucleic acid molecule is a plasmid. Also provided are host cells comprising the nucleic acids.

Other aspects and advantages of these compositions and methods are described further in the following detailed description.

### BRIEF DESCRIPTION OF THE FIGURES

FIG. 1A-FIG. 1B provide an overview of an AAV vector library production (FIG. 1A) and screening, including a depiction of the AAV9 capsid and positions of the amino acids that were altered in the design of the library (FIG. 1B).

FIG. 2 shows a plot of yield rankings for variants in a first-round library screen.

FIG. 3 shows a plot of enrichment of variants in either heart (top) or liver (bottom) for a first-round library screen.

FIG. 4 shows a plot of enrichment of variants in heart vs. liver for a first-round library screen.

FIG. 5 shows a plot of enrichment of variants in heart vs. liver for a first-round library screen. Results for yield score >0.25 relative to AAV9 are shown.

FIG. 6 shows an overview of the AAV9 variants identified in a first-round screen (˜200 AAV9 Gal-binding variants).

FIG. 7A-FIG. 7F provide charts showing positional analysis of variants identified in heart and liver.

FIG. 8 provides an overview of the study design for a second-round AAV vector production and screening.

FIG. 9A and FIG. 9B show enrichment in heart vs. liver in mice. The plot identifies a collection of variants having the XRH motif and which exhibited enhanced heart targeting and liver detargeting.

FIG. 10 show enrichment in heart vs. liver in a non-human primate.

FIG. 11 shows a comparison of enrichment in liver in mouse and NHP. Most variants performed similarly in mouse and NHP liver, whereas AAV9 and its close cousins had a lower RPM in NHP liver than they did in mouse liver.

FIG. 12A and FIG. 12B provide an overview of methods and results for studies assessing galactose binding affinity in vitro to characterize the relative binding of strength of AAV9 variants.

FIG. 13A and FIG. 13B provide results from galactose binding affinity studies.

FIG. 14A-FIG. 14C show relative transduction (DNA) (FIG. 14A) and RNA (FIG. 14B) and protein (FIG. 14C) expression of an eGFP transgene in heart following delivery of vectors having HRH, SRH, and VRH capsids. Vectors with an AAV9 capsid or an AAV9 capsid having the W503A mutation were included as controls.

FIG. 15A-FIG. 15C show relative transduction (DNA) (FIG. 15A) and RNA (FIG. 15B) and protein (FIG. 15C) expression of an eGFP transgene in liver following delivery of vectors having HRH, SRH, and VRH capsids. Vectors with an AAV9 capsid or an AAV9 capsid having the W503A mutation were included as controls.

### DETAILED DESCRIPTION OF THE INVENTION

Novel adeno-associated virus (AAV) capsid proteins are provided herein. In certain embodiments the capsid proteins are characterized by reduced galactose binding, which alters the ability of viral vectors to transduce certain cells and tissue types. In certain embodiments, the capsid protein is a clade F capsid protein with the following motif: Y446X, N470R, and W503H, where the number of the amino acid residues is relative to the vp1 protein of a known clade F vector such as AAV9. Also provided are rAAV that include the capsid proteins described herein. In certain embodiments, the rAAV have improved ability to target cardiac tissue and/or reduced ability to target the liver following administration to a subject. The rAAV may be in a composition used as a gene therapy product, for gene editing, as a vaccine, amongst other suitable uses. Also provided are compositions including nucleic acids that encode the capsid proteins described herein, including host cells for production of a rAAV.

Based on observations that suggest that both liver detargeting and peripheral transduction are dependent upon factors within the combinatorial space of the AAV9 galactose binding site, directed evolution approach was utilized to identify rAAV vectors with novel capsid proteins. A library of AAV variants containing amino acid substitutions at Y446, N470, and W503 was generated and screened through multiple rounds of selection in both mouse and in non-human primates. A family of AAV capsid variants containing a shared amino acid motif, all of which exhibited a reduction in liver transduction with a coincident increase in cardiac transduction, was identified. The studies revealed five amino acid residues that alter galactose binding and the identification of capsid proteins that have altered tissue targeting in vivo. The findings suggest that binding affinity correlates positively with liver transduction in vivo, while non-tryptophan aromatics are favored in vectors that detarget the liver but maintain peripheral transduction.

In certain embodiments, a rAAV having an AAV clade F capsid comprising a mutant galactose binding site is provided. In certain embodiments, the rAAV has enhanced cardiac targeting and decreased liver targeting as compared to its corresponding parental AAV Clade F capsid. The galactose binding site is characterized by (a) any amino acid residue (X) at position 446 (Y446X), (b) an arginine at position 470 (N470R), and (c) a histidine at position 503 (W503H), when residue positions are determined using the residue numbers of SEQ ID NO: 10 (AAV9 vp1) as a reference. The rAAV includes a vector genome comprising a 5′ inverted terminal repeat (ITR) sequence, a coding sequence for a gene product, regulatory sequences operably linked to the coding sequence that direct expression of the gene product, and a 3′ ITR sequence. In certain embodiments, the rAAV comprises a capsid protein that includes (a) H at position 446 (HRH); (b) S at position 446 (SRH); (c) V at position 446 (VRH); (d) G at position 446 (GRH); (e) F at position 446 (FRH); (f) T at position 446 (TRH); or (g) S at position 446 (SRH). In certain embodiments, the parental capsid is an AAV9 capsid. In certain embodiments, the rAAV comprises a capsid protein comprising the amino acid sequence set forth in any one of SEQ ID NOs: 1-9.

In certain embodiments, the rAAV includes additional mutations (insertion(s), deletion(s), substitution(s)). Such additional mutations include those described herein as well as those known in the art. In certain embodiments, the additional mutations improve vector production yields. In certain embodiments, these additional mutations improve targeting or reduce targeting of cells or tissues. In certain embodiments, the additional mutations further improve, e.g., targeting of cardiac cells or tissue.

In certain embodiments, a method for generating an rAAV comprising a mutant capsid derived from a parental AAV capsid having liver specificity is provided. In certain embodiments, the parental AAV capsid is an AAV9, AAVhu68, AAVhu31, AAVhu32, AAVhu95, or AAVhu96 capsid, or a mutant or variant of any of the aforementioned. Accordingly, the rAAV includes a capsid protein having (a) any amino acid residue (X) at position 446 (Y446X), (b) an arginine at position 470 (N470R), and (c) a histidine at position 503 (W503H) when residue positions are determined using the residue numbers of SEQ ID NO: 10 (AAV9 vp1) as a reference. The method comprises culturing a packaging host cell comprising a nucleic acid sequence encoding the mutant AAV Clade F capsid operably linked to regulatory control sequences that direct its expression in the packaging host cell. The packaging cell line further includes a nucleic acid molecule comprising a vector genome comprising a 5′ inverted terminal repeat (ITR) sequence, a coding sequence for a gene product, regulatory sequences operably linked to the coding sequence that direct expression of the gene product, and a 3′ ITR sequence; and helper functions necessary for packaging the vector genome into the mutant Clade F capsid. In certain embodiments, the parental capsid is an AAV9 capsid. In certain embodiments, the nucleic acid sequence encoding the mutant AAV Clade F capsid comprises a sequence that encodes an amino acid sequence set forth in any one of SEQ ID NOs: 1-9.

In certain embodiment, the rAAV or rAAV produced according to the methods provided are useful for delivery of gene product. In certain embodiments, the rAAV has improved ability to target cardiac cells and tissues, in particular relative to the parental clade F capsid. In certain embodiments, the rAAV has reduced ability to transduce liver, in particular relative to the parental clade F capsid, and thus is said to “detarget” liver. Detargeting liver may be advantageous to reduce liver toxicity which may be observed in some cases following delivery of a parental clade F vector. Also provided are compositions, such as a pharmaceutical formulation, that include the described rAAV. In certain embodiments, the method includes reducing liver toxicity associated with delivery of an rAAV vector, wherein a rAAV with a mutated galactose binding site as provided herein is administered to a subject. In certain embodiments, the method includes improving delivery of a gene product to a method for improved delivery of a gene product to cardiac cells or tissue, wherein a rAAV with a mutated galactose binding site as provided herein is administered to a subject. In certain embodiments, the reduction in liver toxicity associated with delivery of an rAAV vector is relative to the parental AAV clade F capsid. In certain embodiments, the improvement in delivery of a gene product to a method for improved delivery of a gene product to cardiac cells or tissue is relative to the parental AAV clade F capsid.

With regard to the following description, it is intended that each of the compositions herein described, is useful, in another embodiment, in the methods of the invention. In addition, it is also intended that each of the compositions herein described as useful in the methods, is, in another embodiment, itself an embodiment of the invention.

### Capsids

In certain embodiments, an rAAV is provided which has a capsid that includes mutations that alter binding to galactose. The altered binding to galactose contributes to enhanced targeting or retargeting of cells and tissues following in vivo delivery. The rAAV provided herein include a capsid with the following: any amino acid residue (X) at position 446 (Y446X), an arginine at position 470 (N470R), and a histidine at position 503 (W503H), where the number of the amino acid residues is determined by reference to the AAV9 vp1 capsid protein (as provided in SEQ ID NO: 10).

In certain embodiments, the rAAV capsid includes mutations that are introduced into or found in a parental capsid. In certain embodiments, the parental capsid is a clade F AAV capsid. In certain embodiments, the parental AAV capsid is an AAV9, AAVhu68, AAVhu31, AAVhu32, AAVhu95, or AAVhu96 capsid, or a mutant or variant of any of the aforementioned. [AAVhu68—See, e.g., US2020/0056159; PCT/US21/55436; SEQ ID NOs: 4 and 5 for nucleic acid sequence encoding a hu68 capsid; SEQ ID NO: 6 for a hu68 vp1 amino acid sequence], AAVhu95 capsid—[See, e.g., U.S. Provisional Application No. 63/251,599, filed Oct. 2, 2201; SEQ ID NOs: 7 and 8 (hu95 vp1 encoding nucleic acid sequences) and SEQ ID NO: 9 (hu95 vp1 amino acid sequence), AAVhu96 capsid—[See, e.g., U.S. Provisional Application No. 63/251,599, filed Oct. 2, 2201; SEQ ID NOs: 10 and 11 (hu96 encoding nucleic acid sequences) and SEQ ID NO: 12 (hu96 vp1 amino acid sequence), AAV9—[See, e.g., U.S. Pat. No. 7,906,111], engineered mutants and variants thereof—[see, e.g., WO2020/200499; WO2003/054197].

As used herein, the term “clade” as it relates to groups of AAV refers to a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence. The Neighbor-Joining algorithm has been described in the literature. See, e.g., M. Nei and S. Kumar, Molecular Evolution and Phylogenetics (Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another clade, or is outside these clades. See, e.g., G Gao, et al, J Virol, 2004 June; 78(12): 6381-6388, which identifies Clades A, B, C, D, E and F, and provides nucleic acid sequences of novel AAV, GenBank Accession Numbers AY530553 to AY530629. See, also, WO 2005/033321.

As used herein, an “AAV9 capsid” is a self-assembled AAV capsid composed of multiple AAV9 vp proteins. The AAV9 vp proteins are typically expressed as alternative splice variants encoded by a nucleic acid sequence which encodes the vp1 amino acid sequence of GenBank accession: AAS99264. These splice variants result in proteins of different length. In certain embodiments, “AAV9 capsid” includes an AAV having an amino acid sequence which is 99% identical to AAS99264 or 99% identical thereto. See, also, WO 2019/168961, published Sep. 6, 2019, including Table G providing the deamidation pattern for AAV9. See, also U.S. Pat. No. 7,906,111 and WO 2005/033321. When specified, “AAV9 variants” may include those described in, e.g., WO2016/049230, U.S. Pat. No. 8,927,514, US 2015/0344911, and U.S. Pat. No. 8,734,809.

An rAAVhu68 is composed of an AAVhu68 capsid and a vector genome. An AAVhu68 capsid is an assembly of a heterogeneous population of vp1, a heterogeneous population of vp2, and a heterogeneous population of vp3 proteins. As used herein when used to refer to vp capsid proteins, the term “heterogeneous” or any grammatical variation thereof, refers to a population consisting of elements that are not the same, for example, having vp1, vp2 or vp3 monomers (proteins) with different modified amino acid sequences. See, also, PCT/US2018/019992, WO 2018/160582, entitled “Adeno-Associated Virus (AAV) Clade F Vector and Uses Therefor”, and which are incorporated herein by reference in its entirety.

In certain embodiments, the rAAV includes an AAV capsid protein that includes any amino acid residue (X) at position 446 (Y446X), an arginine at position 470 (N470R), and a histidine at position 503 (W503H). In certain embodiments, the capsid protein is an AAV9 variant that includes V, S, H, G, F, Y, T, or A residue at position 446, and an arginine at position 470 (N470R) and a histidine at position 503 (W503H) (SEQ ID NO: 9). In certain embodiments, capsid protein is an AAV9 variant that includes any amino acid residue (X) at position 446, an arginine at position 470 (N470R), and a histidine at position 503 (W503H), and shares as least 95%, at least 96%, at least 97%, at least 98%, or at least 99% identity with SEQ IN NO: 10 (AAV9). In certain embodiments, the amino acid capsid protein is an AAV9 variant that has any amino acid residue (X) at position 446, an arginine at position 470 (N470R), and a histidine at position 503 (W503H), and at least 1, 2, 3, 4, 5, 6, 7, 8, 9, or 10 additional amino acid substitutions (relative to SEQ ID NO: 10). In certain embodiments, the capsid protein is an AAVhu68 variant that includes any amino acid residue (X) at position 446, an arginine at position 470 (N470R), and a histidine at position 503 (W503H). In certain embodiments, capsid protein is an AAVhu68 variant that includes any amino acid residue (X) at position 446, an arginine at position 470 (N470R), and a histidine at position 503 (W503H), and shares as least 95%, at least 96%, at least 97%, at least 98%, or at least 99% identity with SEQ IN NO: 11. In certain embodiments, the amino acid capsid protein is an AAVhu68 variant that includes any amino acid residue (X) at position 446, an arginine at position 470 (N470R), and a histidine at position 503 (W503H), and at least 1, 2, 3, 4, 5, 6, 7, 8, 9, or 10 additional amino acid substitutions (relative to SEQ ID NO: 11).

In certain embodiments, the rAAV includes an AAV capsid protein comprising the amino acid sequence set forth in SEQ ID NO: 1 (VRH). In certain embodiments, the rAAV includes an AAV capsid protein comprising an amino acid sequence having V at position 446, an arginine at position 470 (N470R), and a histidine at position 503 (W503H) that shares as least 95%, at least 96%, at least 97%, at least 98%, or at least 99% identity with SEQ IN NO: 10. In certain embodiments, the AAV capsid protein is an AAV9 variant that includes V at position 446, an arginine at position 470 (N470R), and a histidine at position 503 (W503H), and at least 1, 2, 3, 4, 5, 6, 7, 8, 9, or 10 additional amino acid substitutions (relative to SEQ ID NO: 10). In certain embodiments, the rAAV includes an AAV capsid protein comprising an amino acid sequence having a V at position 446, an arginine at position 470 (N470R), and a histidine at position 503 (W503H) that shares as least 95%, at least 96%, at least 97%, at least 98%, or at least 99% identity with SEQ IN NO: 11. In certain embodiments, the capsid protein is an AAVhu68 variant that includes V at position 446, and an arginine at position 470 (N470R) and a histidine at position 503 (W503H), and at least 1, 2, 3, 4, 5, 6, 7, 8, 9, or 10 amino acid substitutions (relative to SEQ ID NO: 11).

In certain embodiments, the rAAV includes an AAV capsid protein comprising the amino acid sequence set forth in SEQ ID NO: 2 (SRH). In certain embodiments, the rAAV includes an AAV capsid protein comprising an amino acid sequence having S at position 446, an arginine at position 470 (N470R), and a histidine at position 503 (W503H) that shares as least 95%, at least 96%, at least 97%, at least 98%, or at least 99% identity with SEQ IN NO: 10. In certain embodiments, the AAV capsid protein is an AAV9 variant that includes S at position 446, an arginine at position 470 (N470R), and a histidine at position 503 (W503H), and at least 1, 2, 3, 4, 5, 6, 7, 8, 9, or 10 additional amino acid substitutions (relative to SEQ ID NO: 10). In certain embodiments, the rAAV includes an AAV capsid protein comprising an amino acid sequence having a S at position 446, an arginine at position 470 (N470R), and a histidine at position 503 (W503H) that shares as least 95%, at least 96%, at least 97%, at least 98%, or at least 99% identity with SEQ IN NO: 11. In certain embodiments, the amino acid capsid protein is an AAVhu68 variant that includes S at position 446, and an arginine at position 470 (N470R) and a histidine at position 503 (W503H), and at least 1, 2, 3, 4, 5, 6, 7, 8, 9, or 10 amino acid substitutions (relative to SEQ ID NO: 11).

In certain embodiments, the rAAV includes an AAV capsid protein comprising the amino acid sequence set forth in SEQ ID NO: 3 (HRH). In certain embodiments, the rAAV includes an AAV capsid protein comprising an amino acid sequence having H at position 446, an arginine at position 470 (N470R), and a histidine at position 503 (W503H) that shares as least 95%, at least 96%, at least 97%, at least 98%, or at least 99% identity with SEQ IN NO: 10. In certain embodiments, the AAV capsid protein is an AAV9 variant that includes H at position 446, an arginine at position 470 (N470R), and a histidine at position 503 (W503H), and at least 1, 2, 3, 4, 5, 6, 7, 8, 9, or 10 additional amino acid substitutions (relative to SEQ ID NO: 10). In certain embodiments, the rAAV includes an AAV capsid protein comprising an amino acid sequence having a H at position 446, an arginine at position 470 (N470R), and a histidine at position 503 (W503H) that shares as least 95%, at least 96%, at least 97%, at least 98%, or at least 99% identity with SEQ IN NO: 11. In certain embodiments, the amino acid capsid protein is an AAVhu68 variant that includes H at position 446, and an arginine at position 470 (N470R) and a histidine at position 503 (W503H), and at least 1, 2, 3, 4, 5, 6, 7, 8, 9, or 10 amino acid substitutions (relative to SEQ ID NO: 11).

In certain embodiments, the rAAV includes an AAV capsid protein comprising the amino acid sequence set forth in SEQ ID NO: 4 (GRH). In certain embodiments, the rAAV includes an AAV capsid protein comprising an amino acid sequence having G at position 446, an arginine at position 470 (N470R), and a histidine at position 503 (W503H) that shares as least 95%, at least 96%, at least 97%, at least 98%, or at least 99% identity with SEQ IN NO: 10. In certain embodiments, the AAV capsid protein is an AAV9 variant that includes G at position 446, an arginine at position 470 (N470R), and a histidine at position 503 (W503H), and at least 1, 2, 3, 4, 5, 6, 7, 8, 9, or 10 additional amino acid substitutions (relative to SEQ ID NO: 10). In certain embodiments, the rAAV includes an AAV capsid protein comprising an amino acid sequence having a G at position 446, an arginine at position 470 (N470R), and a histidine at position 503 (W503H) that shares as least 95%, at least 96%, at least 97%, at least 98%, or at least 99% identity with SEQ IN NO: 11. In certain embodiments, the amino acid capsid protein is an AAVhu68 variant that includes G at position 446, and an arginine at position 470 (N470R) and a histidine at position 503 (W503H), and at least 1, 2, 3, 4, 5, 6, 7, 8, 9, or 10 amino acid substitutions (relative to SEQ ID NO: 11).

In certain embodiments, the rAAV includes an AAV capsid protein comprising the amino acid sequence set forth in SEQ ID NO: 5 (FRH). In certain embodiments, the rAAV includes an AAV capsid protein comprising an amino acid sequence having F at position 446, an arginine at position 470 (N470R), and a histidine at position 503 (W503H) that shares as least 95%, at least 96%, at least 97%, at least 98%, or at least 99% identity with SEQ IN NO: 10. In certain embodiments, the AAV capsid protein is an AAV9 variant that includes F at position 446, an arginine at position 470 (N470R), and a histidine at position 503 (W503H), and at least 1, 2, 3, 4, 5, 6, 7, 8, 9, or 10 additional amino acid substitutions (relative to SEQ ID NO: 10). In certain embodiments, the rAAV includes an AAV capsid protein comprising an amino acid sequence having a F at position 446, an arginine at position 470 (N470R), and a histidine at position 503 (W503H) that shares as least 95%, at least 96%, at least 97%, at least 98%, or at least 99% identity with SEQ IN NO: 11. In certain embodiments, the amino acid capsid protein is an AAVhu68 variant that includes F at position 446, and an arginine at position 470 (N470R) and a histidine at position 503 (W503H), and at least 1, 2, 3, 4, 5, 6, 7, 8, 9, or 10 amino acid substitutions (relative to SEQ ID NO: 11).

In certain embodiments, the rAAV includes an AAV capsid protein comprising the amino acid sequence set forth in SEQ ID NO: 6 (YRH). In certain embodiments, the rAAV includes an AAV capsid protein comprising an amino acid sequence having Y at position 446, an arginine at position 470 (N470R), and a histidine at position 503 (W503H) that shares as least 95%, at least 96%, at least 97%, at least 98%, or at least 99% identity with SEQ IN NO: 10. In certain embodiments, the AAV capsid protein is an AAV9 variant that includes Y at position 446, an arginine at position 470 (N470R), and a histidine at position 503 (W503H), and at least 1, 2, 3, 4, 5, 6, 7, 8, 9, or 10 additional amino acid substitutions (relative to SEQ ID NO: 10). In certain embodiments, the rAAV includes an AAV capsid protein comprising an amino acid sequence having a Y at position 446, an arginine at position 470 (N470R), and a histidine at position 503 (W503H) that shares as least 95%, at least 96%, at least 97%, at least 98%, or at least 99% identity with SEQ IN NO: 11. In certain embodiments, the amino acid capsid protein is an AAVhu68 variant that includes Y at position 446, and an arginine at position 470 (N470R) and a histidine at position 503 (W503H), and at least 1, 2, 3, 4, 5, 6, 7, 8, 9, or 10 amino acid substitutions (relative to SEQ ID NO: 11).

In certain embodiments, the rAAV includes an AAV capsid protein comprising the amino acid sequence set forth in SEQ ID NO: 7 (TRH). In certain embodiments, the rAAV includes an AAV capsid protein comprising an amino acid sequence having T at position 446, an arginine at position 470 (N470R), and a histidine at position 503 (W503H) that shares as least 95%, at least 96%, at least 97%, at least 98%, or at least 99% identity with SEQ IN NO: 10. In certain embodiments, the AAV capsid protein is an AAV9 variant that includes T at position 446, an arginine at position 470 (N470R), and a histidine at position 503 (W503H), and at least 1, 2, 3, 4, 5, 6, 7, 8, 9, or 10 additional amino acid substitutions (relative to SEQ ID NO: 10). In certain embodiments, the rAAV includes an AAV capsid protein comprising an amino acid sequence having a T at position 446, an arginine at position 470 (N470R), and a histidine at position 503 (W503H) that shares as least 95%, at least 96%, at least 97%, at least 98%, or at least 99% identity with SEQ IN NO: 11. In certain embodiments, the amino acid capsid protein is an AAVhu68 variant that includes T at position 446, and an arginine at position 470 (N470R) and a histidine at position 503 (W503H), and at least 1, 2, 3, 4, 5, 6, 7, 8, 9, or 10 amino acid substitutions (relative to SEQ ID NO: 11).

In certain embodiments, the rAAV includes an AAV capsid protein comprising the amino acid sequence set forth in SEQ ID NO: 8 (ARH). In certain embodiments, the rAAV includes an AAV capsid protein comprising an amino acid sequence having A at position 446, an arginine at position 470 (N470R), and a histidine at position 503 (W503H) that shares as least 95%, at least 96%, at least 97%, at least 98%, or at least 99% identity with SEQ IN NO: 10. In certain embodiments, the AAV capsid protein is an AAV9 variant that includes A at position 446, an arginine at position 470 (N470R), and a histidine at position 503 (W503H), and at least 1, 2, 3, 4, 5, 6, 7, 8, 9, or 10 additional amino acid substitutions (relative to SEQ ID NO: 10). In certain embodiments, the rAAV includes an AAV capsid protein comprising an amino acid sequence having an A at position 446, an arginine at position 470 (N470R), and a histidine at position 503 (W503H) that shares as least 95%, at least 96%, at least 97%, at least 98%, or at least 99% identity with SEQ IN NO: 11. In certain embodiments, the amino acid capsid protein is an AAVhu68 variant that includes A at position 446, and an arginine at position 470 (N470R) and a histidine at position 503 (W503H), and at least 1, 2, 3, 4, 5, 6, 7, 8, 9, or 10 amino acid substitutions (relative to SEQ ID NO: 11).

| Variant | Amino Acid Sequence | SEQ ID NO: |
|---|---|---|
| VRH | MAADGYLPDWLEDNLSEGIREWWALKPGAPQPKANQQHQDNA | 1 |
| | RGLVLPGYKYLGPGNGLDKGEPVNAADAAALEHDKAYDQQLK | |
| | AGDNPYLKYNHADAEFQERLKEDTSFGGNLGRAVFQAKKRLLE | |
| | PLGLVEEAAKTAPGKKRPVEQSPQEPDSSAGIGKSGAQPAKKRL | |
| | NFGQTGDTESVPDPQPIGEPPAAPSGVGSLTMASGGGAPVADNN | |
| | EGADGVGSSSGNWHCDSQWLGDRVITTSTRTWALPTYNNHLYK | |
| | QISNSTSGGSSNDNAYFGYSTPWGYFDFNRFHCHFSPRDWQRLI | |
| | NNNWGFRPKRLNFKLFNIQVKEVTDNNGVKTIANNLTSTVQVFT | |
| | DSDYQLPYVLGSAHEGCLPPFPADVFMIPQYGYLTLNDGSQAVG | |
| | RSSFYCLEYFPSQMLRTGNNFQFSYEFENVPFHSSYAHSQSLDRL | |
| | MNPLIDQYLYVLSKTINGSGQNQQTLKFSVAGPSRMAVQGRNYI | |
| | PGPSYRQQRVSTTVTQNNNSEFAHPGASSWALNGRNSLMNPGP | |
| | AMASHKEGEDRFFPLSGSLIFGKQGTGRDNVDADKVMITNEEEI | |
| | KTTNPVATESYGQVATNHQSAQAQAQTGWVQNQGILPGMVWQ | |
| | DRDVYLQGPIWAKIPHTDGNFHPSPLMGGFGMKHPPPQILIKNTP | |
| | VPADPPTAFNKDKLNSFITQYSTGQVSVEIEWELQKENSKRWNP | |
| | EIQYTSNYYKSNNVEFAVNTEGVYSEPRPIGTRYLTRNL | |
| SRH | MAADGYLPDWLEDNLSEGIREWWALKPGAPQPKANQQHQDNA | 2 |
| | RGLVLPGYKYLGPGNGLDKGEPVNAADAAALEHDKAYDQQLK | |
| | AGDNPYLKYNHADAEFQERLKEDTSFGGNLGRAVFQAKKRLLE | |
| | PLGLVEEAAKTAPGKKRPVEQSPQEPDSSAGIGKSGAQPAKKRL | |
| | NFGQTGDTESVPDPQPIGEPPAAPSGVGSLTMASGGGAPVADNN | |
| | EGADGVGSSSGNWHCDSQWLGDRVITTSTRTWALPTYNNHLYK | |
| | QISNSTSGGSSNDNAYFGYSTPWGYFDFNRFHCHFSPRDWQRLI | |
| | NNNWGFRPKRLNFKLFNIQVKEVTDNNGVKTIANNLTSTVQVFT | |
| | DSDYQLPYVLGSAHEGCLPPFPADVFMIPQYGYLTLNDGSQAVG | |
| | RSSFYCLEYFPSQMLRTGNNFQFSYEFENVPFHSSYAHSQSLDRL | |
| | MNPLIDQYLYSLSKTINGSGQNQQTLKFSVAGPSRMAVQGRNYI | |
| | PGPSYRQQRVSTTVTQNNNSEFAHPGASSWALNGRNSLMNPGP | |
| | AMASHKEGEDRFFPLSGSLIFGKQGTGRDNVDADKVMITNEEEI | |
| | KTTNPVATESYGQVATNHQSAQAQAQTGWVQNQGILPGMVWQ | |
| | DRDVYLQGPIWAKIPHTDGNFHPSPLMGGFGMKHPPPQILIKNTP | |
| | VPADPPTAFNKDKLNSFITQYSTGQVSVEIEWELQKENSKRWNP | |
| | EIQYTSNYYKSNNVEFAVNTEGVYSEPRPIGTRYLTRNL | |
| HRH | MAADGYLPDWLEDNLSEGIREWWALKPGAPQPKANQQHQDNA | 3 |
| | RGLVLPGYKYLGPGNGLDKGEPVNAADAAALEHDKAYDQQLK | |
| | AGDNPYLKYNHADAEFQERLKEDTSFGGNLGRAVFQAKKRLLE | |
| | PLGLVEEAAKTAPGKKRPVEQSPQEPDSSAGIGKSGAQPAKKRL | |
| | NFGQTGDTESVPDPQPIGEPPAAPSGVGSLTMASGGGAPVADNN | |
| | EGADGVGSSSGNWHCDSQWLGDRVITTSTRTWALPTYNNHLYK | |
| | QISNSTSGGSSNDNAYFGYSTPWGYFDFNRFHCHFSPRDWQRLI | |
| | NNNWGFRPKRLNFKLFNIQVKEVTDNNGVKTIANNLTSTVQVFT | |
| | DSDYQLPYVLGSAHEGCLPPFPADVFMIPQYGYLTLNDGSQAVG | |
| | RSSFYCLEYFPSQMLRTGNNFQFSYEFENVPFHSSYAHSQSLDRL | |
| | MNPLIDQYLYHLSKTINGSGQNQQTLKFSVAGPSRMAVQGRNYI | |
| | PGPSYRQQRVSTTVTQNNNSEFAHPGASSWALNGRNSLMNPGP | |
| | AMASHKEGEDRFFPLSGSLIFGKQGTGRDNVDADKVMITNEEEI | |
| | KTTNPVATESYGQVATNHQSAQAQAQTGWVQNQGILPGMVWQ | |
| | DRDVYLQGPIWAKIPHTDGNFHPSPLMGGFGMKHPPPQILIKNTP | |
| | VPADPPTAFNKDKLNSFITQYSTGQVSVEIEWELQKENSKRWNP | |
| | EIQYTSNYYKSNNVEFAVNTEGVYSEPRPIGTRYLTRNL | |
| GRH | MAADGYLPDWLEDNLSEGIREWWALKPGAPQPKANQQHQDNA | 4 |
| | RGLVLPGYKYLGPGNGLDKGEPVNAADAAALEHDKAYDQQLK | |
| | AGDNPYLKYNHADAEFQERLKEDTSFGGNLGRAVFQAKKRLLE | |
| | PLGLVEEAAKTAPGKKRPVEQSPQEPDSSAGIGKSGAQPAKKRL | |
| | NFGQTGDTESVPDPQPIGEPPAAPSGVGSLTMASGGGAPVADNN | |
| | EGADGVGSSSGNWHCDSQWLGDRVITTSTRTWALPTYNNHLYK | |
| | QISNSTSGGSSNDNAYFGYSTPWGYFDFNRFHCHFSPRDWQRLI | |
| | NNNWGFRPKRLNFKLFNIQVKEVTDNNGVKTIANNLTSTVQVFT | |
| | DSDYQLPYVLGSAHEGCLPPFPADVFMIPQYGYLTLNDGSQAVG | |
| | RSSFYCLEYFPSQMLRTGNNFQFSYEFENVPFHSSYAHSQSLDRL | |
| | MNPLIDQYLYGLSKTINGSGQNQQTLKFSVAGPSRMAVQGRNYI | |
| | PGPSYRQQRVSTTVTQNNNSEFAHPGASSWALNGRNSLMNPGP | |
| | AMASHKEGEDRFFPLSGSLIFGKQGTGRDNVDADKVMITNEEEI | |
| | KTTNPVATESYGQVATNHQSAQAQAQTGWVQNQGILPGMVWQ | |
| | DRDVYLQGPIWAKIPHTDGNFHPSPLMGGFGMKHPPPQILIKNTP | |
| | VPADPPTAFNKDKLNSFITQYSTGQVSVEIEWELQKENSKRWNP | |
| | EIQYTSNYYKSNNVEFAVNTEGVYSEPRPIGTRYLTRNL | |
| FRH | MAADGYLPDWLEDNLSEGIREWWALKPGAPQPKANQQHQDNA | 5 |
| | RGLVLPGYKYLGPGNGLDKGEPVNAADAAALEHDKAYDQQLK | |
| | AGDNPYLKYNHADAEFQERLKEDTSFGGNLGRAVFQAKKRLLE | |
| | PLGLVEEAAKTAPGKKRPVEQSPQEPDSSAGIGKSGAQPAKKRL | |
| | NFGQTGDTESVPDPQPIGEPPAAPSGVGSLTMASGGGAPVADNN | |
| | EGADGVGSSSGNWHCDSQWLGDRVITTSTRTWALPTYNNHLYK | |
| | QISNSTSGGSSNDNAYFGYSTPWGYFDFNRFHCHFSPRDWQRLI | |
| | NNNWGFRPKRLNFKLFNIQVKEVTDNNGVKTIANNLTSTVQVFT | |
| | DSDYQLPYVLGSAHEGCLPPFPADVFMIPQYGYLTLNDGSQAVG | |
| | RSSFYCLEYFPSQMLRTGNNFQFSYEFENVPFHSSYAHSQSLDRL | |
| | MNPLIDQYLYFLSKTINGSGQNQQTLKFSVAGPSRMAVQGRNYI | |
| | PGPSYRQQRVSTTVTQNNNSEFAHPGASSWALNGRNSLMNPGP | |
| | AMASHKEGEDRFFPLSGSLIFGKQGTGRDNVDADKVMITNEEEI | |
| | KTTNPVATESYGQVATNHQSAQAQAQTGWVQNQGILPGMVWQ | |
| | DRDVYLQGPIWAKIPHTDGNFHPSPLMGGFGMKHPPPQILIKNTP | |
| | VPADPPTAFNKDKLNSFITQYSTGQVSVEIEWELQKENSKRWNP | |
| | EIQYTSNYYKSNNVEFAVNTEGVYSEPRPIGTRYLTRNL | |
| YRH | MAADGYLPDWLEDNLSEGIREWWALKPGAPQPKANQQHQDNA | 6 |
| | RGLVLPGYKYLGPGNGLDKGEPVNAADAAALEHDKAYDQQLK | |
| | AGDNPYLKYNHADAEFQERLKEDTSFGGNLGRAVFQAKKRLLE | |
| | PLGLVEEAAKTAPGKKRPVEQSPQEPDSSAGIGKSGAQPAKKRL | |
| | NFGQTGDTESVPDPQPIGEPPAAPSGVGSLTMASGGGAPVADNN | |
| | EGADGVGSSSGNWHCDSQWLGDRVITTSTRTWALPTYNNHLYK | |
| | QISNSTSGGSSNDNAYFGYSTPWGYFDFNRFHCHFSPRDWQRLI | |
| | NNNWGFRPKRLNFKLFNIQVKEVTDNNGVKTIANNLTSTVQVFT | |
| | DSDYQLPYVLGSAHEGCLPPFPADVFMIPQYGYLTLNDGSQAVG | |
| | RSSFYCLEYFPSQMLRTGNNFQFSYEFENVPFHSSYAHSQSLDRL | |
| | MNPLIDQYLYYLSKTINGSGQNQQTLKFSVAGPSRMAVQGRNYI | |
| | PGPSYRQQRVSTTVTQNNNSEFAHPGASSWALNGRNSLMNPGP | |
| | AMASHKEGEDRFFPLSGSLIFGKQGTGRDNVDADKVMITNEEEI | |
| | KTTNPVATESYGQVATNHQSAQAQAQTGWVQNQGILPGMVWQ | |
| | DRDVYLQGPIWAKIPHTDGNFHPSPLMGGFGMKHPPPQILIKNTP | |
| | VPADPPTAFNKDKLNSFITQYSTGQVSVEIEWELQKENSKRWNP | |
| | EIQYTSNYYKSNNVEFAVNTEGVYSEPRPIGTRYLTRNL | |
| TRH | MAADGYLPDWLEDNLSEGIREWWALKPGAPQPKANQQHQDNA | 7 |
| | RGLVLPGYKYLGPGNGLDKGEPVNAADAAALEHDKAYDQQLK | |
| | AGDNPYLKYNHADAEFQERLKEDTSFGGNLGRAVFQAKKRLLE | |
| | PLGLVEEAAKTAPGKKRPVEQSPQEPDSSAGIGKSGAQPAKKRL | |
| | NFGQTGDTESVPDPQPIGEPPAAPSGVGSLTMASGGGAPVADNN | |
| | EGADGVGSSSGNWHCDSQWLGDRVITTSTRTWALPTYNNHLYK | |
| | QISNSTSGGSSNDNAYFGYSTPWGYFDFNRFHCHFSPRDWQRLI | |
| | NNNWGFRPKRLNFKLFNIQVKEVTDNNGVKTIANNLTSTVQVFT | |
| | DSDYQLPYVLGSAHEGCLPPFPADVFMIPQYGYLTLNDGSQAVG | |
| | RSSFYCLEYFPSQMLRTGNNFQFSYEFENVPFHSSYAHSQSLDRL | |
| | MNPLIDQYLYTLSKTINGSGQNQQTLKFSVAGPSRMAVQGRNYI | |
| | PGPSYRQQRVSTTVTQNNNSEFAHPGASSWALNGRNSLMNPGP | |
| | AMASHKEGEDRFFPLSGSLIFGKQGTGRDNVDADKVMITNEEEI | |
| | KTTNPVATESYGQVATNHQSAQAQAQTGWVQNQGILPGMVWQ | |
| | DRDVYLQGPIWAKIPHTDGNFHPSPLMGGFGMKHPPPQILIKNTP | |
| | VPADPPTAFNKDKLNSFITQYSTGQVSVEIEWELQKENSKRWNP | |
| | EIQYTSNYYKSNNVEFAVNTEGVYSEPRPIGTRYLTRNL | |
| ARH | MAADGYLPDWLEDNLSEGIREWWALKPGAPQPKANQQHQDNA | 8 |
| | RGLVLPGYKYLGPGNGLDKGEPVNAADAAALEHDKAYDQQLK | |
| | AGDNPYLKYNHADAEFQERLKEDTSFGGNLGRAVFQAKKRLLE | |
| | PLGLVEEAAKTAPGKKRPVEQSPQEPDSSAGIGKSGAQPAKKRL | |
| | NFGQTGDTESVPDPQPIGEPPAAPSGVGSLTMASGGGAPVADNN | |
| | EGADGVGSSSGNWHCDSQWLGDRVITTSTRTWALPTYNNHLYK | |
| | QISNSTSGGSSNDNAYFGYSTPWGYFDFNRFHCHFSPRDWQRLI | |
| | NNNWGFRPKRLNFKLFNIQVKEVTDNNGVKTIANNLTSTVQVFT | |
| | DSDYQLPYVLGSAHEGCLPPFPADVFMIPQYGYLTLNDGSQAVG | |
| | RSSFYCLEYFPSQMLRTGNNFQFSYEFENVPFHSSYAHSQSLDRL | |
| | MNPLIDQYLYALSKTINGSGQNQQTLKFSVAGPSRMAVQGRNYI | |
| | PGPSYRQQRVSTTVTQNNNSEFAHPGASSWALNGRNSLMNPGP | |
| | AMASHKEGEDRFFPLSGSLIFGKQGTGRDNVDADKVMITNEEEI | |
| | KTTNPVATESYGQVATNHQSAQAQAQTGWVQNQGILPGMVWQ | |
| | DRDVYLQGPIWAKIPHTDGNFHPSPLMGGFGMKHPPPQILIKNTP | |
| | VPADPPTAFNKDKLNSFITQYSTGQVSVEIEWELQKENSKRWNP | |
| | EIQYTSNYYKSNNVEFAVNTEGVYSEPRPIGTRYLTRNL | |
| XRH | MAADGYLPDWLEDNLSEGIREWWALKPGAPQPKANQQHQDNA | 9 |
| | RGLVLPGYKYLGPGNGLDKGEPVNAADAAALEHDKAYDQQLK | |
| | AGDNPYLKYNHADAEFQERLKEDTSFGGNLGRAVFQAKKRLLE | |
| | PLGLVEEAAKTAPGKKRPVEQSPQEPDSSAGIGKSGAQPAKKRL | |
| | NFGQTGDTESVPDPQPIGEPPAAPSGVGSLTMASGGGAPVADNN | |
| | EGADGVGSSSGNWHCDSQWLGDRVITTSTRTWALPTYNNHLYK | |
| | QISNSTSGGSSNDNAYFGYSTPWGYFDFNRFHCHFSPRDWQRLI | |
| | NNNWGFRPKRLNFKLFNIQVKEVTDNNGVKTIANNLTSTVQVFT | |
| | DSDYQLPYVLGSAHEGCLPPFPADVFMIPQYGYLTLNDGSQAVG | |
| | RSSFYCLEYFPSQMLRTGNNFQFSYEFENVPFHSSYAHSQSLDRL | |
| | MNPLIDQYLYXLSKTINGSGQNQQTLKFSVAGPSRMAVQGRNYI | |
| | PGPSYRQQRVSTTVTQNNNSEFAHPGASSWALNGRNSLMNPGP | |
| | AMASHKEGEDRFFPLSGSLIFGKQGTGRDNVDADKVMITNEEEI | |
| | KTTNPVATESYGQVATNHQSAQAQAQTGWVQNQGILPGMVWQ | |
| | DRDVYLQGPIWAKIPHTDGNFHPSPLMGGFGMKHPPPQILIKNTP | |
| | VPADPPTAFNKDKLNSFITQYSTGQVSVEIEWELQKENSKRWNP | |
| | EIQYTSNYYKSNNVEFAVNTEGVYSEPRPIGTRYLTRNL | |

In certain embodiments, an rAAV is provided which has a modified capsid protein having at least an exogenous peptide from the N- x- (T/I/V/A)-(K/R) targeting motif. In other embodiments, other viral vectors may be generated having one or more exogenous targeting peptides from the N- x- (T/I/V/A)-(K/R) motif (which may be same or different, or combinations thereof) in an exposed capsid protein to modulate and/or alter the targeting specificity of the viral vector as compared to the parental vector. In certain embodiments, a composition is provided which is useful for targeting an endothelial cell. The composition is a mutant capsid comprising at least one exogenous targeting peptide comprising: an amino acid sequence of N- x- (T/I/V/A)-(K/R) (SEQ ID NO: 47) optionally flanked at the amino terminus and/or the carboxy terminus of the motif by two amino acids to seven amino acids, and optionally further conjugated to a nanoparticle, a second molecule, or a viral capsid protein.

In certain embodiments, the targeting peptide comprises one of the following sequences with optional linking sequences:

(SEQ ID NO: 14)
(a) SSNTVKLTSGH;
(SEQ ID NO: 12)
(b) EFSSNTVKLTS;
(SEQ ID NO: 18)
(c) GGVLTNIARGEYMRGG;
(SEQ ID NO: 17)
(d) GGIEINATRAGTNLGG;
(SEQ ID NO: 13)
(e) GGSSNTVKLTSGHGG;
(SEQ ID NO: 16)
(f) IEINATRAGTNL;
or
(SEQ ID NO: 15)
(g) SANFIKPTSY.

See PCT/US2021/061312, filed Dec. 1, 2021, entitled “NOVEL COMPOSITIONS WITH TISSUE-SPECIFIC TARGETING MOTIFS AND COMPOSITIONS CONTAINING SAME,” which is incorporated herein by reference in its entirety

In certain embodiments, an rAAV is provided which has a modified capsid having at least an exogenous peptide from the Y-G/A/R/K-Y/H-GNPA-T/R/H-RYFD-V/K (SEQ ID NO: 28) core targeting motif. In certain embodiments, an rAAV may have one or more exogenous targeting peptides from the Y-G/A/R/K-Y/H-GNPA-T/R/H-RYFD-V/K (SEQ ID NO: 28) core targeting motif (which may be same or different, or combinations thereof) in an exposed capsid protein to modulate and/or alter the targeting specificity of the viral vector as compared to the parental vector.

In certain embodiment, an rAAV is provided which is useful for targeting a brain cell. The composition is a capsid protein comprising at least one exogenous targeting peptide comprising: a core amino acid sequence of YGYGNPATRYFDV (SEQ ID NO: 20) optionally flanked at its amino terminus and/or carboxy terminus of the core sequence by two amino acids to seven amino acids, and optionally further conjugated to a nanoparticle, a second molecule, or a viral capsid protein. In certain embodiments, an rAAV having a capsid which is useful for targeting a brain cell. The capsid comprises at least one exogenous targeting peptide comprising: a core amino acid sequence of YGYGNPATRYFDV (SEQ ID NO: 20) optionally flanked at its amino terminus and/or carboxy terminus of the core sequence by two amino acids to seven amino acids, and optionally further conjugated to a nanoparticle, a second molecule, or a viral capsid protein. The targeting peptide comprises the following core amino acid sequence with optional linking sequences:

(SEQ ID NO: 25)
(a) YGYGNPARRYFDV;
(SEQ ID NO: 26)
(b) YGYGNPAHRYFDV;
(SEQ ID NO: 27)
(c) YGYGNPATRYFDK;
(SEQ ID NO: 21)
(d) YAYGNPATRYFDV;
(SEQ ID NO: 22)
(e) YKYGNPATRYFDV;
(SEQ ID NO: 23)
(f) YRYGNPATRYFDV;
or
(SEQ ID NO: 24)
(g) YGHGNPATRYFDV.

See U.S. Provisional Application No. 63/178,881, filed Apr. 23, 2021, entitled “NOVEL COMPOSITIONS WITH BRAIN-SPECIFIC TARGETING MOTIFS AND COMPOSITIONS CONTAINING SAME,” which is incorporated herein by reference in its entirety.

In certain embodiments, an rAAV is provided which has been modified to include a peptide insertion that improves targeting of muscle tissues, including skeletal and/or cardiac tissue (i.e., improved binding to receptors expressed on cardiac cells). In certain embodiments, the peptide insertion includes an RGD motif. See, e.g. See PCT/EP2019/076958, filed Oct. 4, 2019, entitled “PEPTIDE-MODIFIED HYBRID RECOMBINANT ADENO-ASSOCIATED VIRUS SEROTYPE BETWEEN AAV9 AND AAVrh74 WITH REDUCED LIVER TROPISM AND INCREASED MUSCLE TRANSDUCTION,” which is incorporated herein by reference in its entirety.

A targeting peptide as described above may be inserted into a hypervariable loop (HVR) VIII at any suitable location. For example, the peptide is inserted with linkers of various lengths between amino acids 588 and 589 (Q-A) of the AAV9 capsid protein, based on the numbering of the AAV9 VP1 amino acid sequence: SEQ ID NO: 10. See, also, WO 2019/168961, published Sep. 6, 2019, including Table G providing the deamidation pattern for AAV9 and WO 2020/160582, filed Sep. 7, 2018. The amino acid residue locations are identical in AAVhu68 (SEQ ID NO: 11). However, another site may be selected within HVRVIII. Alternatively, another exposed loop HVR (e.g., HVRIV) may be selected for the insertion. Comparable HVR regions may be selected in other capsids. In certain embodiments, the location for the HVRVIII and HVRIV is determined using an algorithm and/or alignment technique as described in U.S. Pat. No. 9,737,618 B2 (column 15, lines 3-23), and U.S. Pat. No. 10,308,958 B2 (column 15, line 46—column 16, line 6), which are incorporated herein by reference in its entirety. In certain embodiments, the targeting peptide may be inserted into a hypervariable loop HVRVIII as described in U.S. Provisional Patent Application No. 63/119,863, filed Dec. 1, 2020, which is incorporated herein by reference.

In certain embodiments, a nucleic acid comprising a sequence that encodes a novel capsid protein described herein is provide. In certain embodiments, the encoded amino acid sequence comprises any one of SEQ ID NOs: 1 to 9. In certain embodiments, the encoded amino acid sequence shares at least 95%, at least 96%, at least 97%, at least 98%, or at least 99% identity with any one of SEQ ID NOs: 1 to 9. In certain embodiments, the encoded amino acid sequence is an AAV clade F capsid protein having any amid acid residue at position 446, and an arginine at position 470 (N470R) and a histidine at position 503 (W503H), and at least 1, 2, 3, 4, 5, 6, 7, 8, 9, or 10 additional amino acid substitutions (relative to SEQ ID NO: 10). In certain embodiments, the nucleic acid is a plasmid.

It is within the skill in the art to design nucleic acid sequences encoding AAV capsid proteins, including DNA (genomic or cDNA), or RNA (e.g., mRNA). Such nucleic acid sequences may be codon-optimized for expression in a selected system (i.e., cell type) and can be designed by various methods. This optimization may be performed using methods which are available on-line (e.g., GeneArt), published methods, or a company which provides codon optimizing services, e.g., DNA2.0 (Menlo Park, CA). One codon optimizing method is described, e.g., in International Patent Publication No. WO 2015/012924, which is incorporated by reference herein in its entirety. See also, e.g., US Patent Publication No. 2014/0032186 and US Patent Publication No. 2006/0136184.

As used herein, “encoded amino acid sequence” refers to the amino acid which is predicted based on the translation of a known DNA codon of a referenced nucleic acid sequence being translated to an amino acid. The following table illustrates DNA codons and twenty common amino acids, showing both the single letter code (SLC) and three letter code (3LC).

Inverse table for the standard genetic code (compressed using IUPAC notation)

| Amino | | | Amino | | |
|---|---|---|---|---|---|
| acid | DNA codons | Compressed | acid | DNA codons | Compressed |
| Ala, A | GCT, GCC, GCA, | GCN | Ile, I | ATT, ATC, ATA | ATH |
| | GCG | | | | |
| Arg, R | CGT, CGC, CGA, | CGN, AGR; | Leu, L | CTT, CTC, CTA, | CTN, TTR; |
| | CGG; AGA, AGG | or | | CTG; TTA, TTG | or |
| | | CGY, MGR | | | CTY, YTR |
| Asn, N | AAT, AAC | AAY | Lys, K | AAA, AAG | AAR |
| Asp, D | GAT, GAC | GAY | Met, M | ATG | |
| Asn or | AAT, AAC; GAT, | RAY | Phe, F | TTT, TTC | TTY |
| Asp, B | GAC | | Pro, P | CCT, CCC, CCA, | CCN |
| | | | | CCG | |
| Cys, C | TGT, TGC | TGY | Ser, S | TCT, TCC, TCA, | TCN, AGY |
| | | | | TCG; AGT, AGC | |
| Gln, Q | CAA, CAG | CAR | Thr, T | ACT, ACC, | ACN |
| | | | | ACA, ACG | |
| Glu, E | GAA, GAG | GAR | Trp, W | TGG | |
| Gln or | CAA, CAG; | SAR | Tyr, Y | TAT, TAC | TAY |
| Glu, Z | GAA, GAG | | Val, V | GTT, GTC, GTA, | GTN |
| | | | | GTG | |
| Gly, G | GGT, GGC, GGA, | GGN | STOP | TAA, TGA, TAG | TRA, TAR |
| | GGG | | | | |
| His, H | CAT, CAC | CAY | | | |

| | |
|---|---|
| START | ATG |

### Expression Cassette and Vectors

Vector genomic sequences which are packaged into an AAV capsid and delivered to a host cell are typically composed of, at a minimum, a transgene and its regulatory sequences, and AAV inverted terminal repeats (ITRs). Both single-stranded AAV and self-complementary (sc) AAV are encompassed with the rAAV. The transgene is a nucleic acid coding sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In one embodiment, the ITRs are from an AAV different than that supplying a capsid. In one embodiment, the ITR sequences from AAV2. However, ITRs from other AAV sources may be selected. A shortened version of the 5′ ITR, termed AITR, has been described in which the D-sequence and terminal resolution site (trs) are deleted. In certain embodiments, the vector genome includes a shortened AAV2 ITR of 130 base pairs, wherein the external A elements is deleted. Without wishing to be bound by theory, it is believed that the shortened ITR is reverts back to the wild type length of 145 base pairs during vector DNA amplification using the internal (A′) element as a template. In other embodiments, full-length AAV 5′ and 3′ ITRs are used. Where the source of the ITRs is from AAV2 and the AAV capsid is from another AAV source, the resulting vector may be termed pseudotyped. However, other configurations of these elements may be suitable.

In addition to the major elements identified above for the recombinant AAV vector, the vector also includes conventional control elements necessary which are operably linked to the transgene in a manner which permits its transcription, translation and/or expression in a cell transfected with the plasmid vector or infected with the virus produced by the invention.

The regulatory control elements typically contain a promoter sequence as part of the expression control sequences, e.g., located between the selected 5′ ITR sequence and the coding sequence. Constitutive promoters, regulatable promoters [see, e.g., WO 2011/126808 and WO 2013/04943], tissue specific promoters, or a promoter responsive to physiologic cues may be used may be utilized in the vectors described herein.

Examples of constitutive promoters suitable for controlling expression of the therapeutic products include, but are not limited to chickenβ-actin (CB) promoter, CB7 promoter, human cytomegalovirus (CMV) promoter, ubiquitin C promoter (UbC), the early and late promoters of simian virus 40 (SV40), U6 promoter, metallothionein promoters, EF1α promoter, ubiquitin promoter, hypoxanthine phosphoribosyl transferase (HPRT) promoter, dihydrofolate reductase (DHFR) promoter (Scharfmann et al., Proc. Natl. Acad. Sci. USA 88:4626-4630 (1991), adenosine deaminase promoter, phosphoglycerol kinase (PGK) promoter, pyruvate kinase promoter phosphoglycerol mutase promoter, the β-actin promoter (Lai et al., Proc. Natl. Acad. Sci. USA 86: 10006-10010 (1989)), the long terminal repeats (LTR) of Moloney Leukemia Virus and other retroviruses, the thymidine kinase promoter of Herpes Simplex Virus and other constitutive promoters known to those of skill in the art. Examples of tissue- or cell-specific promoters suitable for use in the present invention include, but are not limited to, endothelin-I (ET-I) and Flt-I, which are specific for endothelial cells, FoxJ1 (that targets ciliated cells). Other examples of tissue specific promoters suitable for use in the present invention include, but are not limited to, liver-specific promoters. Examples of liver-specific promoters may include, e.g., thyroid hormone-binding globulin (TBG), albumin, Miyatake et al., (1997) J. Virol., 71:5124 32; hepatitis B virus core promoter, Sandig et al., (1996) Gene Ther., 3:1002 9; or human alpha 1-antitrypsin, phosphoenolpyruvate carboxykinase (PECK), or alpha fetoprotein (AFP), Arbuthnot et al., (1996) Hum. Gene Ther., 7:1503 14).

In certain embodiments, the promoter is a tissue-specific (e.g., neuron-specific) promoter. In certain embodiments, a suitable promoter may include without limitation, an elongation factor 1 alpha (EF1 alpha) promoter (see, e.g., Kim D W et al, Use of the human elongation factor 1 alpha promoter as a versatile and efficient expression system. Gene. 1990 Jul. 16; 91(2):217-23), a Synapsin 1 promoter (see, e.g., Kugler S et al, Human synapsin 1 gene promoter confers highly neuron-specific long-term transgene expression from an adenoviral vector in the adult rat brain depending on the transduced area. Gene Ther. 2003 February; 10(4):337-47), a shorted synapsin promoter, a neuron-specific enolase (NSE) promoter (see, e.g., Kim J et al, Involvement of cholesterol-rich lipid rafts in interleukin-6-induced neuroendocrine differentiation of LNCaP prostate cancer cells. Endocrinology. 2004 February; 145(2):613-9. Epub 2003 Oct. 16), or a CB6 promoter (see, e.g., Large-Scale Production of Adeno-Associated Viral Vector Serotype-9 Carrying the Human Survival Motor Neuron Gene, Mol Biotechnol. 2016 January; 58(1):30-6. doi: 10.1007/s12033-015-9899-5). Preferably, such promoters are of human origin.

In certain embodiments, the promoter is a cardiac promoter. In certain embodiments, the promoter is a cardiac troponin T (cTNT), desmin (DES), alpha-myosin heavy chain (α-MHC), or myosin light chain 2 (MLC-2) promoter. See also, Pacak, C. A., et al., Tissue specific promoters improve specificity of AAV9 mediated transgene expression following intra-vascular gene delivery in neonatal mice, Genetic Vaccines and Therapy 2008, 6:13. In certain embodiments, the expression cassette comprises a promoter which is a chicken cardiac Troponin T promoter (also referred to as chicken TnT or chTnT).

Inducible promoters suitable for controlling expression of the therapeutic product include promoters responsive to exogenous agents (e.g., pharmacological agents) or to physiological cues. These response elements include, but are not limited to a hypoxia response element (HRE) that binds HIF-Iα and β, a metal-ion response element such as described by Mayo et al. (1982, Cell 29:99-108); Brinster et al. (1982, Nature 296:39-42) and Searle et al. (1985, Mol. Cell. Biol. 5:1480-1489); or a heat shock response element such as described by Nouer et al. (in: Heat Shock Response, ed. Nouer, L., CRC, Boca Raton, Fla., ppI67-220, 1991).

In one embodiment, expression of the gene product is controlled by a regulatable promoter that provides tight control over the transcription of the sequence encoding the gene product, e.g., a pharmacological agent, or transcription factors activated by a pharmacological agent or in alternative embodiments, physiological cues. Promoter systems that are non-leaky and that can be tightly controlled are preferred. Examples of regulatable promoters which are ligand-dependent transcription factor complexes that may be used in the invention include, without limitation, members of the nuclear receptor superfamily activated by their respective ligands (e.g., glucocorticoid, estrogen, progestin, retinoid, ecdysone, and analogs and mimetics thereof) and rTTA activated by tetracycline. In one aspect of the invention, the gene switch is an EcR-based gene switch. Examples of such systems include, without limitation, the systems described in U.S. Pat. Nos. 6,258,603, 7,045,315, U.S. Published Patent Application Nos. 2006/0014711, 2007/0161086, and International Published Application No. WO 01/70816. Examples of chimeric ecdysone receptor systems are described in U.S. Pat. No. 7,091,038, U.S. Published Patent Application Nos. 2002/0110861, 2004/0033600, 2004/0096942, 2005/0266457, and 2006/0100416, and International Published Application Nos. WO 01/70816, WO 02/066612, WO 02/066613, WO 02/066614, WO 02/066615, WO 02/29075, and WO 2005/108617, each of which is incorporated by reference in its entirety. An example of a non-steroidal ecdysone agonist-regulated system is the RheoSwitch® Mammalian Inducible Expression System (New England Biolabs, Ipswich, MA).

Still other promoter systems may include response elements including but not limited to a tetracycline (tet) response element (such as described by Gossen & Bujard (1992, Proc. Natl. Acad. Sci. USA 89:5547-551); or a hormone response element such as described by Lee et al. (1981, Nature 294:228-232); Hynes et al. (1981, Proc. Natl. Acad. Sci. USA 78:2038-2042); Klock et al. (1987, Nature 329:734-736); and Israel & Kaufman (1989, Nucl. Acids Res. 17:2589-2604) and other inducible promoters known in the art. Using such promoters, expression of the soluble hACE2 construct can be controlled, for example, by the Tet-on/off system (Gossen et al., 1995, Science 268:1766-9; Gossen et al., 1992, Proc. Natl. Acad. Sci. USA., 89(12):5547-51); the TetR-KRAB system (Urrutia R., 2003, Genome Biol., 4(10):231; Deuschle U et al., 1995, Mol Cell Biol. (4):1907-14); the mifepristone (RU486) regulatable system (Geneswitch; Wang Y et al., 1994, Proc. Natl. Acad. Sci. USA., 91(17):8180-4; Schillinger et al., 2005, Proc. Natl. Acad. Sci. USA. 102(39):13789-94); and the humanized tamoxifen-dep regulatable system (Roscilli et al., 2002, Mol. Ther. 6(5):653-63).

In another aspect, the gene switch is based on heterodimerization of FK506 binding protein (FKBP) with FKBP rapamycin associated protein (FRAP) and is regulated through rapamycin or its non-immunosuppressive analogs. Examples of such systems, include, without limitation, the ARGENT™ Transcriptional Technology (ARIAD Pharmaceuticals, Cambridge, Mass.) and the systems described in U.S. Pat. Nos. 6,015,709, 6,117,680, 6,479,653, 6,187,757, and 6,649,595, U.S. Publication No. 2002/0173474, U.S. Publication No. 200910100535, U.S. Pat. Nos. 5,834,266, 7,109,317, 7,485,441, 5,830,462, 5,869,337, 5,871,753, 6,011,018, 6,043,082, 6,046,047, 6,063,625, 6,140,120, 6,165,787, 6,972,193, 6,326,166, 7,008,780, 6,133,456, 6,150,527, 6,506,379, 6,258,823, 6,693,189, 6,127,521, 6,150,137, 6,464,974, 6,509,152, 6,015,709, 6,117,680, 6,479,653, 6,187,757, 6,649,595, 6,984,635, 7,067,526, 7,196,192, 6,476,200, 6,492,106, WO 94/18347, WO 96/20951, WO 96/06097, WO 97/31898, WO 96/41865, WO 98/02441, WO 95/33052, WO 99110508, WO 99110510, WO 99/36553, WO 99/41258, WO 01114387, ARGENT™ Regulated Transcription Retrovirus Kit, Version 2.0 (9109102), and ARGENT™ Regulated Transcription Plasmid Kit, Version 2.0 (9109/02), each of which is incorporated herein by reference in its entirety. The Ariad system is designed to be induced by rapamycin and analogs thereof referred to as “rapalogs”. Examples of suitable rapamycins are provided in the documents listed above in connection with the description of the ARGENT™ system. In one embodiment, the molecule is rapamycin [e.g., marketed as Rapamune™ by Pfizer]. In another embodiment, a rapalog known as AP21967 [ARIAD] is used. Examples of these dimerizer molecules that can be used in the present invention include, but are not limited to rapamycin, FK506, FK1012 (a homodimer of FK506), rapamycin analogs (“rapalogs”) which are readily prepared by chemical modifications of the natural product to add a “bump” that reduces or eliminates affinity for endogenous FKBP and/or FRAP. Examples of rapalogs include, but are not limited to such as AP26113 (Ariad), AP1510 (Amara, J. F., et al., 1997, Proc Natl Acad Sci USA, 94(20): 10618-23) AP22660, AP22594, AP21370, AP22594, AP23054, AP1855, AP1856, AP1701, AP1861, AP1692 and AP1889, with designed ‘bumps’ that minimize interactions with endogenous FKBP. Still other rapalogs may be selected, e.g., AP23573 [Merck]. In certain embodiments, rapamycin or a suitable analog may be delivered locally to the AAV-transfected cells of the nasopharynx. This local delivery may be by intranasal injection, topically to the cells via bolus, cream, or gel. See, US Patent Application US 2019/0216841 A1, which is incorporated herein by reference.

Other suitable enhancers include those that are appropriate for a desired target tissue indication. In one embodiment, the expression cassette comprises one or more expression enhancers. In one embodiment, the expression cassette contains two or more expression enhancers. These enhancers may be the same or may differ from one another. For example, an enhancer may include a CMV immediate early enhancer. This enhancer may be present in two copies which are located adjacent to one another. Alternatively, the dual copies of the enhancer may be separated by one or more sequences. In still another embodiment, the expression cassette further contains an intron, e.g., the chicken beta-actin intron. Other suitable introns include those known in the art, e.g., such as are described in WO 2011/126808. Examples of suitable polyA sequences include, e.g., rabbit beta globin (rBG), SV40, SV50, bovine growth hormone (bGH), human growth hormone, and synthetic polyAs. Optionally, one or more sequences may be selected to stabilize mRNA. An example of such a sequence is a modified WPRE sequence, which may be engineered upstream of the polyA sequence and downstream of the coding sequence (see, e.g., MA Zanta-Boussif, et al, Gene Therapy (2009) 16: 605-619).

An AAV viral vector may include multiple transgenes. In certain embodiments, the transgene may be used to correct or ameliorate gene deficiencies, which may include deficiencies in which normal genes are expressed at less than normal levels or deficiencies in which the functional gene product is not expressed. Alternatively, the transgene may provide a product to a cell which is not natively expressed in the cell type or in the host. A preferred type of transgene sequence encodes a therapeutic protein or polypeptide which is expressed in a host cell. The invention further includes using multiple transgenes. In certain situations, a different transgene may be used to encode each subunit of a protein, or to encode different peptides or proteins. This is desirable when the size of the DNA encoding the protein subunit is large, e.g., for an immunoglobulin, the platelet-derived growth factor, or a dystrophin protein. In certain situations, a different transgene may be used to encode each subunit of a protein (e.g., an immunoglobulin domain, an immunoglobulin heavy chain, an immunoglobulin light chain). In one embodiment, a cell produces the multi-subunit protein following infected/transfection with the virus containing each of the different subunits. In another embodiment, different subunits of a protein may be encoded by the same transgene. An IRES is desirable when the size of the DNA encoding each of the subunits is small, e.g., the total size of the DNA encoding the subunits and the IRES is less than five kilobases. As an alternative to an IRES, the DNA may be separated by sequences encoding a 2A peptide, which self-cleaves in a post-translational event. See, e.g., ML Donnelly, et al, (January 1997) J. Gen. Virol., 78(Pt 1):13-21; S. Furler, S et al, (June 2001) Gene Ther., 8(11):864-873; H. Klump, et al., (May 2001) Gene Ther., 8(10):811-817. This 2A peptide is significantly smaller than IRES, making it well suited for use when space is a limiting factor. More often, when the transgene is large, consists of multi-subunits, or two transgenes are co-delivered, rAAV carrying the desired transgene(s) or subunits are co-administered to allow them to concatamerize in vivo to form a single vector genome. In such an embodiment, a first AAV may carry an expression cassette which expresses a single transgene and a second AAV may carry an expression cassette which expresses a different transgene for co-expression in the host cell. However, the selected transgene may encode any biologically active product or other product, e.g., a product desirable for study.

In addition to the elements identified above for the expression cassette, the vector also includes conventional control elements which are operably linked to the coding sequence in a manner which permits transcription, translation and/or expression of the encoded product (e.g., UBE3A construct, gene replacement therapy in Angelman mouse models; see, U.S. Provisional Patent Application No. 63/119,860, filed Dec. 1, 2020, which is incorporated herein by reference) in a cell transfected with the plasmid vector or infected with the virus produced by the invention. Examples of other suitable transgenes are provided herein.

Expression control sequences include appropriate enhancer; transcription factor; transcription terminator; promoter; efficient RNA processing signals such as splicing and polyadenylation (polyA) signals; sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence); sequences that enhance protein stability; and when desired, sequences that enhance secretion of the encoded product. In one embodiment, the regulatory sequences are selected such that the total rAAV vector genome is about 2.0 to about 5.5 kilobases in size. In one embodiment, it is desirable that the rAAV vector genome approximate the size of the native AAV genome. Thus, in one embodiment, the regulatory sequences are selected such that the total rAAV vector genome is about 4.7 kb in size. In another embodiment, the total rAAV vector genome is less about 5.2 kb in size. The size of the vector genome may be manipulated based on the size of the regulatory sequences including the promoter, enhancer, intron, poly A, etc. See, Wu et al, Mol Ther, January 2010 18(1):80-6, which is incorporated herein by reference.

Thus, in one embodiment, an intron is included in the vector. Suitable introns include chicken beta-actin intron, the human beta globin IVS2 (Kelly et al, Nucleic Acids Research, 43(9):4721-32 (2015)); the Promega chimeric intron (Almond, B. and Schenborn, E. T. A Comparison of pCI-neo Vector and pcDNA4/HisMax Vector); and the hFIX intron. Various introns suitable herein are known in the art and include, without limitation, those found at bpg.utoledo.edu/˜afedorov/lab/eid.html, which is incorporated herein by reference. See also, Shepelev V., Fedorov A. Advances in the Exon-Intron Database. Briefings in Bioinformatics 2006, 7: 178-185, which is incorporated herein by reference.

rAAV Vector Production

Nucleic acid for use in producing AAV viral vectors (e.g., an rAAV) and the expression cassettes including the same can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art.

Methods of preparing AAV-based vectors (e.g., having an AAV9 or another AAV capsid) are known. See, e.g., US Published Patent Application No. 2007/0036760 (Feb. 15, 2007), which is incorporated by reference herein. The sequences of any of the AAV capsids provided herein can be readily generated using a variety of techniques. Suitable production techniques are well known to those of skill in the art. See, e.g., Sambrook et al, Molecular Cloning: A Laboratory Manual, Cold Spring Harbor Press (Cold Spring Harbor, NY). Alternatively, peptides can also be synthesized by the well-known solid phase peptide synthesis methods (Merrifield, (1962) J. Am. Chem. Soc., 85:2149; Stewart and Young, Solid Phase Peptide Synthesis (Freeman, San Francisco, 1969) pp. 27-62). These methods may involve, e.g., culturing a host cell which contains a nucleic acid sequence encoding an AAV capsid; a functional rep gene; a minigene composed of, at a minimum, AAV inverted terminal repeats (ITRs) and a transgene; and sufficient helper functions to permit packaging of the minigene into the AAV capsid protein. These and other suitable production methods are within the knowledge of those of skill in the art and are not a limitation of the present invention.

The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

The rAAV described herein are particularly well suited for gene delivery for therapeutic purposes. Further, the compositions described may also be used for production of a desired gene product in vitro. For in vitro production, a desired product (e.g., a protein) may be obtained from a desired culture following transfection of host cells with an rAAV containing the molecule encoding the desired product and culturing the cell culture under conditions which permit expression. The expressed product may then be purified and isolated, as desired. Suitable techniques for transfection, cell culturing, purification, and isolation are known to those of skill in the art. Methods for generating and isolating AAVs suitable for use as vectors are known in the art. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J. Gene Med. 10:717-733; and the references cited below, each of which is incorporated herein by reference in its entirety. For packaging a transgene into virions, the ITRs are the only AAV components required in cis in the same construct as the nucleic acid molecule containing the expression cassettes. The cap and rep genes can be supplied in trans.

In one embodiment, the expression cassettes described herein are engineered into a genetic element (e.g., a shuttle plasmid) which transfers the immunoglobulin construct sequences carried thereon into a packaging host cell for production a viral vector. In one embodiment, the selected genetic element may be delivered to an AAV packaging cell by any suitable method, including transfection, electroporation, liposome delivery, membrane fusion techniques, high velocity DNA-coated pellets, viral infection and protoplast fusion. Stable AAV packaging cells can also be made. Alternatively, the expression cassettes may be used to generate a viral vector other than AAV, or for production of mixtures of antibodies in vitro. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, NY (2012).

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product. These empty capsids are non-functional to transfer the gene of interest to a host cell.

The recombinant AAV described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid; a functional rep gene; an expression cassette composed of, at a minimum, AAV inverted terminal repeats (ITRs) and a transgene; and sufficient helper functions to permit packaging of the expression cassette into the AAV capsid protein. Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, vectors are manufactured in a suitable cell culture (e.g., HEK 293 cells). Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV genome and the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. See, also, U.S. Provisional Patent Application No. 63/371,597, filed Aug. 16, 2022, entitled “Scalable Methods for Producing rAAV with Packaged Vector Genomes, and U.S. Provisional Patent Application No. 63/371,592, filed Aug. 16, 2022, entitled “Scalable Methods for Downstream Purification of Recombinant Adeno-associated Virus”, both incorporated by reference in their entirety.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector. A two-step affinity chromatography purification at high salt concentration followed anion exchange resin chromatography are used to purify the vector drug product and to remove empty capsids. These methods are described in more detail in International Patent Application No. PCT/US2016/065970, filed Dec. 9, 2016, entitled “Scalable Purification Method for AAV9”, which is incorporated by reference. Purification methods for AAV8, International Patent Application No. PCT/US2016/065976, filed Dec. 9, 2016, and rh10, International Patent Application No. PCT/US16/066013, filed Dec. 9, 2016, entitled “Scalable Purification Method for AAVrh10”, also filed Dec. 11, 2015, and for AAV1, International Patent Application No. PCT/US2016/065974, filed Dec. 9, 2016, for “Scalable Purification Method for AAV1”, filed Dec. 11, 2015, are all incorporated by reference herein.

To calculate empty and full particle content, vp3 band volumes for a selected sample (e.g., in examples herein an iodixanol gradient-purified preparation where number of GC=number of particles) are plotted against GC particles loaded. The resulting linear equation (y=mx+c) is used to calculate the number of particles in the band volumes of the test article peaks. The number of particles (pt) per 20 L loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and x 100 gives the percentage of empty particles.

Generally, methods for assaying for empty capsids and AAV vector particles with packaged genomes have been known in the art. See, e.g., Grimm et al., Gene Therapy (1999) 6:1322-1330; and Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e., SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

Additionally, another example of measuring empty to full particle ratio is also known in the art. Sedimentation velocity, as measured in an analytical ultracentrifuge (AUC) can detect aggregates, other minor components as well as providing good quantitation of relative amounts of different particle species based upon their different sedimentation coefficients. This is an absolute method based on fundamental units of length and time, requiring no standard molecules as references. Vector samples are loaded into cells with 2-channel charcoal-epon centerpieces with 12 mm optical path length. The supplied dilution buffer is loaded into the reference channel of each cell. The loaded cells are then placed into an AN-60Ti analytical rotor and loaded into a Beckman-Coulter ProteomeLab XL-I analytical ultracentrifuge equipped with both absorbance and RI detectors. After full temperature equilibration at 20° C. the rotor is brought to the final run speed of 12,000 rpm. A₂₈₀ scans are recorded approximately every 3 minutes for ˜5.5 hours (110 total scans for each sample). The raw data is analyzed using the c(s) method and implemented in the analysis program SEDFIT. The resultant size distributions are graphed and the peaks integrated. The percentage values associated with each peak represent the peak area fraction of the total area under all peaks and are based upon the raw data generated at 280 nm; many labs use these values to calculate empty: full particle ratios. However, because empty and full particles have different extinction coefficients at this wavelength, the raw data can be adjusted accordingly. The ratio of the empty particle and full monomer peak values both before and after extinction coefficient-adjustment is used to determine the empty-full particle ratio.

In one aspect, an optimized q-PCR method is used which utilizes a broad-spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000-fold) and subjected to TaqMan analysis as described in the standard assay. Quantification also can be done using ViroCyt or flow cytometry.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

### Pharmaceutical Compositions

Provided herein are compositions containing at least one rAAV or rAAV stock and an optional carrier, excipient and/or preservative.

In certain embodiments, a composition may contain at least a second, different rAAV or rAAV stock. This second vector or vector stock may vary from the first by having a different AAV capsid and/or a different vector genome. In certain embodiments, a composition as described herein may contain a different vector expressing an expression cassette as described herein, or another active component (e.g., an antibody construct, another biologic, and/or a small molecule drug).

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions.

The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered transgenes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Suitably, the final formulation is adjusted to a physiologically acceptable pH, e.g., the pH may be in the range of pH 6 to 9, or pH 6.5 to 8.5, or pH 7 to 7.8. As the pH of the cerebrospinal fluid is about 7.28 to about 7.32, for intrathecal delivery, a pH within this range may be desired; whereas for intravenous delivery, a pH of 6.8 to about 7.2 may be desired. However, other pHs within the broadest ranges and these subranges may be selected for other routes of delivery. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

In certain embodiments, the formulation buffer is phosphate-buffered saline (PBS) with total salt concentration of 200 mM, 0.001% (w/v) pluronic F68 (Final Formulation Buffer, FFB).

In certain embodiments, the composition comprises a viral vector (i.e., rAAV vector). The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. In certain embodiments, the vectors are formulated for delivery via intranasal delivery devices. In certain embodiments, vectors are formulated for aerosol delivery devices, e.g., via a nebulizer or through other suitable devices. In certain embodiment, vectors are formulated for intrathecal delivery. In some embodiments, intrathecal delivery encompasses an injection into the spinal canal, e.g., the subarachnoid space. In some embodiments, other delivery route may be selected, e.g., intracranial, intranasal, intracistemal, intracerebrospinal fluid delivery, among other suitable direct or systemic routes, i.e., Ommaya reservoir. In certain embodiments, vectors are formulated for intravenous delivery. Other conventional and pharmaceutically acceptable routes of administration include, but are not limited to, direct delivery to a desired organ (e.g., lung), oral inhalation, intratracheal, intraarterial, intraocular, intravenous, intramuscular, subcutaneous, intradermal, and other parenteral routes of administration. In one embodiment, the vector is administered intranasally using intranasal mucosal atomization device (LMA® MAD Nasal™-MAD110). In another embodiment the vector is administered intrapulmonary in nebulized form using Vibrating Mesh Nebulizer (Aerogen® Solo) or MADgic™ Laryngeal Mucosal Atomizer. Routes of administration may be combined, if desired. Routes of administration and utilization of which for delivering rAAV vectors are also described in the following published US Patent Applications, the contents of each of which is incorporated herein by reference in its entirety: US 2018/0155412A1, US 2018/0243416A1, US 2014/0031418 A1, and US 2019/0216841A1.

Dosages of the viral vector will depend primarily on factors such as the condition being treated, the age, weight and health of the patient, and may thus vary among patients. For example, a therapeutically effective human dosage of the viral vector is generally in the range of from about 25 to about 1000 microliters to about 5 mL of aqueous suspending liquid containing doses of from about 10⁹ to 4×10¹⁴ GC of AAV vector. The dosage will be adjusted to balance the therapeutic benefit against any side effects and such dosages may vary depending upon the therapeutic application for which the recombinant vector is employed. The levels of expression of the transgene can be monitored to determine the frequency of dosage resulting in viral vectors, preferably AAV vectors containing the minigene. Optionally, dosage regimens similar to those described for therapeutic purposes may be utilized for immunization using the compositions of the invention.

The replication-defective virus compositions can be formulated in dosage units to contain an amount of replication-defective virus that is in the range of about 10⁹ GC to about 10¹⁶ GC (to treat an average subject of 70 kg in body weight) including all integers or fractional amounts within the range, and preferably 10¹² GC to 10¹⁴ GC for a human patient. In one embodiment, the compositions are formulated to contain at least 10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 10¹³, 2×10¹³ 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 10¹⁰ to about 10¹² GC per dose including all integers or fractional amounts within the range. In one embodiment, for human application the dose can range from 10⁹ to about 7×10¹³ GC per dose including all integers or fractional amounts within the range. In one embodiment, for human application the dose ranges from 6.25×10¹² GC to 5.00×10¹³ GC. In a further embodiment, the dose is about 6.25×10¹² GC, about 1.25×10¹³ GC, about 2.50×10¹³ GC, or about 5.00×10¹³ GC. In certain embodiment, the dose is divided into one half thereof equally and administered to each nostril. In certain embodiments, for human application the dose ranges from 6.25×10¹² GC to 5.00×10¹³ GC administered as two aliquots of 0.2 ml per nostril for a total volume delivered in each subject of 0.8 ml.

These above doses may be administered in a variety of volumes of carrier, excipient or buffer formulation, ranging from about 25 to about 1000 microliters, or higher volumes, including all numbers within the range, depending on the size of the area to be treated, the viral titer used, the route of administration, and the desired effect of the method. In one embodiment, the volume of carrier, excipient or buffer is at least about 25 μL. In one embodiment, the volume is about 50 μL. In another embodiment, the volume is about 75 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 225 μL. In yet another embodiment, the volume is about 250 μL. In yet another embodiment, the volume is about 275 μL. In yet another embodiment, the volume is about 300 μL. In yet another embodiment, the volume is about 325 μL. In another embodiment, the volume is about 350 μL. In another embodiment, the volume is about 375 μL. In another embodiment, the volume is about 400 μL. In another embodiment, the volume is about 450 μL. In another embodiment, the volume is about 500 μL. In another embodiment, the volume is about 550 μL. In another embodiment, the volume is about 600 μL. In another embodiment, the volume is about 650 μL. In another embodiment, the volume is about 700 μL. In another embodiment, the volume is between about 700 and 1000 μL.

In one embodiment, the viral constructs may be delivered in doses of from at least about least 1×10⁹ GCs to about 1×10¹⁵, or about 1×10¹¹ to 5×10¹³ GC. Suitable volumes for delivery of these doses and concentrations may be determined by one of skill in the art. For example, volumes of about 1 μL. to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage will be adjusted to balance the therapeutic benefit against any side effects and such dosages may vary depending upon the therapeutic application for which the recombinant vector is employed.

The compositions according to the present invention may comprise a pharmaceutically acceptable carrier, such as defined above. Suitably, the compositions described herein comprise an effective amount of one or more AAV suspended in a pharmaceutically suitable carrier and/or admixed with suitable excipients designed for delivery to the subject via injection, osmotic pump, intrathecal catheter, or for delivery by another device or route. In one example, the composition is formulated for intrathecal delivery. An effective amount may be determined based on an animal model, rather than a human patient.

As used herein, the terms “intrathecal delivery” or “intrathecal administration” refer to a route of administration via an injection into the spinal canal, more specifically into the subarachnoid space so that it reaches the cerebrospinal fluid (CSF). Intrathecal delivery may include lumbar puncture, intraventricular (including intracerebroventricular (ICV)), suboccipital/intracisternal, and/or C1-2 puncture. For example, material may be introduced for diffusion throughout the subarachnoid space by means of lumbar puncture. In another example, injection may be into the cisterna magna (i.e., intra cisterna magna, or ICM). In certain embodiments, the intrathecal administration is performed as described in US Patent Publication No. 2018-0339065 A1, published Nov. 29, 2019, which is incorporated herein by reference in its entirety. In certain embodiments, the CNS administration is performed using Ommaya Reservoir (also referred to as Ommaya device or Ommaya system).

As used herein, the terms “intracisternal delivery” or “intracisternal administration” refer to a route of administration for drugs directly into the cerebrospinal fluid of the cisterna magna cerebellomedularis, more specifically via a suboccipital puncture or by direct injection into the cisterna magna or via permanently positioned tube.

In one embodiment, a frozen composition is provided which contains an rAAV in a buffer solution as described herein, in frozen form. Optionally, one or more surfactants (e.g., Pluronic F68), stabilizers or preservatives is present in this composition. Suitably, for use, a composition is thawed and titrated to the desired dose with a suitable diluent, e.g., sterile saline or a buffered saline.

In one embodiment, the compositions described herein are used in preparing medicaments for treating a cardiac disorder or disease.

### Methods and Uses

In one aspect, provided herein are methods for treating a human subject diagnosed with cardiac disease (e.g., cardiomyopathy).

The method comprises administering to a subject a suspension of a vector or an rAAV as described herein. In one embodiment, the method comprises administering to a subject a suspension of a rAAV as described herein in a formulation buffer at a dose of about 1×10⁹ genome copies (GC)/kg to about 1×10¹⁴ GC/kg. In a further embodiment, the rAAV is formulated at 3×10¹³ GC/kg.

The methods and compositions described herein may be used for treatment of any of the stages of cardiomyopathy. In certain embodiments, the patient is an infant, a toddler, or the patient is from 3 years to 6 years of age, from 3 years to 12 years of age, from 3 years to 18 years of age, from 3 years to 20 years of age. In certain embodiments, patients are older than 18 years of age. In certain embodiments, the patient is about 20 to 60. In certain embodiments, the patient is about 40 to 50. In certain embodiment, patients are older than 60 years of age.

In certain embodiments, the methods and compositions may be used for treatment of mitochondrial cardiomyopathy associated with Barth Syndrome. Barth Syndrome is a rare, X-linked recessive disorder characterized by a loss of function mutation in TAZ gene (i.e., amenable gene therapy). Bart Syndrome is associated with pediatric onset cardiomyopathy (i.e., by age 5) with neutropenia, mild mitochondrial myopathy (skeletal muscle weakness), and mild intellectual impairment. See also, Sabbah, H. N., Barth syndrome cardiomyopathy: targeting the mitochondria with elamipretide, Heart Failure Reviews (2021) 26:237-253, which is incorporated herein by reference in its entirety.

In certain embodiments, the methods and compositions may be used in treatment of autosomal dominant form of long-QT syndrome caused by a loss-of-function and partial dominant negative mutations in KCNQ1 gene (i.e., amenable to gene replacement or knockdown/replace approach). The autosomal dominant form of long-QT syndrome is associated with syncope and sudden cardiac death usually occurring during exercise or emotional stress, and many patients remain at-risk despite standard of care (beta blockers, cardiac sympathetic denervation) and require Implantable Cardioverter Defibrillator (ICD). See also, Huang H., et al., Mechanisms of KCNQ1 channel dysfunction in long QT syndrome involving voltage sensor domain mutations, Sci. Adv. 2018, 4:1-12, epub Mar. 7, 2018, which is incorporated herein by reference in its entirety.

In certain embodiments, the methods and compositions may be used in treatment of hypertrophic cardiomyopathy. In certain embodiments, the methods and compositions may be used in treatment of hypertrophic cardiomyopathy caused by loss-of-function mutations in the MYBPC3 gene (i.e., amenable to gene therapy). See also, Mearini G., et al., Mybpc3 gene therapy for neonatal cardiomyopathy enables long-term disease prevention in mice, Nature Communication, 2014, 5:5515, epub Dec. 2, 2014, which is incorporated herein by reference in its entirety.

In certain embodiments, the methods and compositions may be used in treatment of long WT syndrome type 2 caused by a loss-of-function mutation in hERG (Kv11.1; also, Kv11.1 voltage-gated potassium channel) gene. See also, Curran M E., et al., A Molecular Basis for Cardiac Arrhythmia: HERG Mutations Cause Long QT Syndrome, Cell, Voi. 80, 795-803, Mar. 10, 1995, and Hylten-Cavallius, L., et al., Patients With Long-QT Syndrome Caused by Impaired hERG-Encoded Kv11.1 Potassium Channel Have Exaggerated Endocrine Pancreatic and Incretin Function Associated With Reactive Hypoglycemia, Circulation, 2017; 135:1705-1719, which are incorporated herein by reference in their entirety.

In certain embodiments, the methods and compositions may be used for treatment of LMNA cardiomyopathy or a disease caused by loss-of-function mutation in the LMNA gene. See also, Kang, S., et al., Laminopathies; Mutations on single gene and various human genetic diseases, BMB Rep. 2018; 51(7): 327-337, which is incorporated herein by reference in its entirety.

In certain embodiments, the methods and compositions may be used for treatment of heart failure, ischemia-reperfusion injury, myocardial infarction, ventricular remodeling, or a disease associated with extracellular superoxide dismutase 3 (SOD3 or EcSOD). See also, US Patent Application Publication No. US20130136729A1, which is incorporated herein by reference in its entirety.

In certain embodiments, the methods and compositions may be used for treatment of myocardial infarction, reduced ejection fraction of the heart or a disease associated with myc transcription factor, cyclin T 1 and cyclin-dependent kinase 9 (CDK9). See also, International Patent Application Publication No. WO2020/165603A1, which is incorporated herein by reference in its entirety.

In certain embodiments, the methods and compositions may be used for treatment of heart failure, or heart tissue damage, or degeneration, or a disease associated with cyclin A2 protein. See also, International Patent Application Publication No. WO2020/051296A1, which is incorporated herein by reference in its entirety.

In certain embodiments, the methods and compositions may be used for treatment of dilated cardiomyopathy (DCM), a heart failure, a cardiac fibrosis, a heart inflammation, an ischemic heart disease, a myocardial infarction, an ischemic/reperfusion (I/R) related injuries, a transverse aortic constriction, or a disease associated with YY1 or BMP7 protein. See also, International Patent Application Publication No. WO2021/021021A1, which is incorporated herein by reference in its entirety.

In certain embodiments, the methods and compositions may be used for treatment of dilated cardiomyopathy or a disease associated with cardiac Apoptosis Repressor with Caspase Recruitment Domain (cARC). See also, International Patent Application Publication No. WO2021/016126A1, which is incorporated herein by reference in its entirety.

Symptoms of cardiomyopathy or a disease associated with a mutation in a LMNA gene, KCNQ1 gene, MYBPC3 gene, TAZ gene, or hERG gene include atrioventricular (AV) conduction block, atrial fibrillation, arrhythmia including atrial arrhythmia such as atrial flutter and atrial tachycardia, and ventricular arrhythmias including sustained ventricular tachycardias, and ventricular fibrillation (VF) and/or heart failure.

In certain embodiments, the methods and compositions described herein may be used to ameliorate one or more symptoms of cardiomyopathy including increased average life span, and/or reduction in progression towards heart failure.

In certain embodiments, the rAAV includes a vector genome comprising a transgene that encodes a therapeutic protein for treatment of a cardiomyopathy and symptoms thereof, such as, e.g., potassium voltage-gated channel subfamily Q member 1 protein (KCNQ1 gene), cardiac myosin binding protein C (MYBPC3 gene), tafazzin (TAZ), Kv11.1 voltage-gated potassium channel protein (hERG gene), Lamin A (LMNA gene).

In certain embodiments, co-therapies or co-treatments may be utilized, which comprise co-administration with another active agent. In certain embodiments, the co-therapy may further comprise administration of beta blockers, angiotensin-converting enzyme (ACE) inhibitors, diuretics. Diuretic agent used may be acetazolamine (Diamox) or other suitable diuretics. In some embodiments, the diuretic agent is administered at the time of gene therapy administration. In some embodiments, the diuretic agent is administered prior to gene therapy administration. In some, embodiments the diuretic agent is administered where the volume of injection is 3 mL. In certain embodiments, the co-treatment may further comprise implantable cardioverter defibrillators (ICD), pacemakers (PM) and/or cardiac resynchronization therapy (CRT).

Optionally, an immunosuppressive co-therapy may be used in a subject in need. Immunosuppressants for such co-therapy include, but are not limited to, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor- (CD25-) or CD3-directed antibodies, anti-IL-2 antibodies, ciclosporin, tacrolimus, sirolimus, IFNβ, IFN-γ, an opioid, or TNF-α (tumor necrosis factor-alpha) binding agent. In certain embodiments, the immunosuppressive therapy may be started 0, 1, 2, 3, 4, 5, 6, 7, or more days prior to or after the gene therapy administration. Such immunosuppressive therapy may involve administration of one, two or more drugs (e.g., glucocorticoids, prednelisone, micophenolate mofetil (MMF) and/or sirolimus (i.e., rapamycin)). Such immunosuppressive drugs may be administrated to a subject in need once, twice or for more times at the same dose or an adjusted dose. Such therapy may involve co-administration of two or more drugs, the (e.g., prednelisone, micophenolate mofetil (EMF) and/or sirolimus (i.e., rapamycin)) on the same day. One or more of these drugs may be continued after gene therapy administration, at the same dose or an adjusted dose. Such therapy may be for about 1 week (7 days), about 60 days, or longer, as needed. In certain embodiments, a tacrolimus-free regimen is selected.

In one embodiment, the rAAV as described herein is administrated once to the subject in need. In another embodiment, the rAAV is administrated more than once to the subject in need.

“Patient” or “subject”, as used herein interchangeably, means a male or female mammalian animal, including a human, a veterinary or farm animal, a domestic animal or pet, and animals normally used for clinical research. In one embodiment, the subject of these methods and compositions is a human patient. In one embodiment, the subject of these methods and compositions is a male or female human patient.

As used herein when used to refer to vp capsid proteins, the term “heterogeneous” or any grammatical variation thereof, refers to a population consisting of elements that are not the same, for example, having vp1, vp2 or vp3 monomers (proteins) with different modified amino acid sequences. SEQ ID NO: 10 provides the encoded amino acid sequence of the AAV9 vp1 protein. SEQ ID NO: 11 provides the encoded amino acid sequence of the AAVhu68 vp1 protein. The term “heterogeneous” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine-glycine (N-G) pairs and optionally further comprising other deamidated amino acids, wherein the deamidation results in an amino acid change and other optional modifications.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine-glycine pairs. Unless otherwise specified, highly deamidated refers to at least 45% deamidated, at least 50% deamidated, at least 60% deamidated, at least 65% deamidated, at least 70%, at least 75%, at least 80%, at least 85%, at least 90%, at least 95%, 97%, 99%, up to about 100% deamidated at a referenced amino acid position, as compared to the predicted amino acid sequence at the reference amino acid position. Such percentages may be determined using 2D-gel, mass spectrometry techniques, or other suitable techniques.

As used herein, a “stock” of rAAV refers to a population of rAAV generated from the same capsid coding sequence in an AAV production system. A variety of production systems, including but not limited to those described herein may be selected. The rAAV stock may include a heterogeneous population of rAAV, which the rAAV have been packaged in AAV capsids expressed from a single AAV capsid coding sequence, but which include heterogeneous subpopulations of rAAV having capsids with deamidation patterns characteristic of the AAV type and production system. See, e.g., WO 2019/168961, published Sep. 6, 2019, including Table G providing the deamidation pattern for AAV9 and WO 2020/160582, filed Sep. 7, 2018. See, also, e.g., WO 2020/223231, published Nov. 5, 2020 (rh91, including table with deamidation pattern), U.S. Provisional Patent Application No. 63/065,616, filed Aug. 14, 2020, and U.S. Provisional Patent Application No. 63/109,734, filed Nov. 4, 2020.

The compositions described herein may be used in a regimen involving co-administration of other active agents. Any suitable method or route can be used to administer such other agents. Routes of administration include, for example, systemic, oral, intravenous, intraperitoneal, subcutaneous, or intramuscular administration. Optionally, the AAV compositions described herein may also be administered by one of these routes.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

As used herein, the term “operably linked” refers to both expression control sequences that are contiguous with the gene of interest and expression control sequences that act in trans or at a distance to control the gene of interest.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”-containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

A “recombinant AAV” or “rAAV” is a DNAse-resistant viral particle containing two elements, an AAV capsid and a vector genome containing at least non-AAV coding sequences packaged within the AAV capsid. In certain embodiments, the capsid contains about 60 proteins composed of vp1 proteins, vp2 proteins, and vp3 proteins, which self-assemble to form the capsid. Unless otherwise specified, “recombinant AAV” or “rAAV” may be used interchangeably with the phrase “rAAV vector”. The rAAV is a “replication-defective virus” or “viral vector”, as it lacks any functional AAV rep gene or functional AAV cap gene and cannot generate progeny. In certain embodiments, the only AAV sequences are the AAV inverted terminal repeat sequences (ITRs), typically located at the extreme 5′ and 3′ ends of the vector genome in order to allow the gene and regulatory sequences located between the ITRs to be packaged within the AAV capsid.

The term “nuclease-resistant” indicates that the AAV capsid has assembled around the expression cassette which is designed to deliver a transgene to a host cell and protects these packaged genomic sequences from degradation (digestion) during nuclease incubation steps designed to remove contaminating nucleic acids which may be present from the production process.

As used herein, the term “host cell” may refer to the packaging cell line in which the rAAV is produced and includes a plasmid comprising an nucleotide sequence that encodes an AAV capsid described herein. In the alternative, the term “host cell” may refer to the target cell in which expression of the transgene is desired.

As used herein, a “cardiac cell” refers to cardiac tissue cells including but not limited to heart cells, cardiac muscle cells (cardiomyocyte), conduction cells, fibroblasts, endothelial cells, smooth muscle cells and peri-vascular cells.

As used herein, a “vector genome” refers to the nucleic acid sequence packaged inside the rAAV capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s), and an AAV 3′ ITR. In certain embodiments, the ITRs are from AAV2, a different source AAV than the capsid, or other than full-length ITRs may be selected. In still other embodiments, longer or shorter AAV ITRs may be selected. In certain embodiments, the ITRs are from the same AAV source as the AAV which provides the rep function during production or a trans-complementing AAV. Further, other ITRs may be used. Further, the vector genome contains regulatory sequences which direct expression of the gene products. Suitable components of a vector genome are discussed in more detail herein. The vector genome is sometimes referred to herein as the “minigene”.

In certain embodiments, non-viral genetic elements used in manufacture of a rAAV, will be referred to as vectors (e.g., production vectors). In certain embodiments, these vectors are plasmids, but the use of other suitable genetic elements is contemplated. Such production plasmids may encode sequences expressed during rAAV production, e.g., AAV capsid or rep proteins required for production of a rAAV, which are not packaged into the rAAV. Alternatively, such a production plasmid may carry the vector genome which is packaged into the rAAV.

As used herein, a “parental capsid” refers to a non-mutated or a non-modified rAAV capsid. In certain embodiments, the parental capsid includes any naturally occurring AAV capsids comprising a wild-type genome encoding for capsid proteins (i.e., vp proteins), wherein the capsid proteins direct the AAV transduction and/or tissue-specific tropism.

As used herein, the terms “target cell” and “target tissue” can refer to any cell or tissue which is intended to be transduced by the subject AAV vector. The term may refer to any one or more of muscle, liver, lung, airway epithelium, central nervous system, neurons, eye (ocular cells), or heart. In one embodiment, the target tissue is heart.

As used herein, a “variant capsid” or a “variant AAV” or “variant AAV capsid” refers to a rAAV or capsid protein that been modified or mutated, for example to include substitutions at position 446, 470, and/or 503 or to include an insertion of a tissue-specific targeting peptide. A parental capsid may in some instances include a capsid protein that is further modified to include substitutions at position 446, 470, and/or 503 as described herein.

As used herein, an “expression cassette” refers to a nucleic acid molecule which comprises a biologically useful nucleic acid sequence (e.g., a gene cDNA encoding a protein, enzyme or other useful gene product, mRNA, etc.) and regulatory sequences operably linked thereto which direct or modulate transcription, translation, and/or expression of the nucleic acid sequence and its gene product. Such regulatory sequences typically include, e.g., one or more of a promoter, an enhancer, an intron, a Kozak sequence, a polyadenylation sequence, and a TATA signal. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region (3′ UTR) comprising a polyadenylation site, among other elements. In certain embodiments, the regulatory sequences are operably linked to the nucleic acid sequence of a gene product, wherein the regulatory sequences are separated from nucleic acid sequence of a gene product by an intervening nucleic acid sequence, i.e., 5′-untranslated regions (5′UTR). In certain embodiments, the expression cassette comprises nucleic acid sequence of one or more of gene products. In some embodiments, the expression cassette can be a monocistronic or a bicistronic expression cassette. In other embodiments, the term “transgene” refers to one or more DNA sequences from an exogenous source which are inserted into a target cell. Typically, such an expression cassette can be used for generating a viral vector and contains the coding sequence for the gene product described herein flanked by packaging signals of the viral genome and other expression control sequences such as those described herein. In certain embodiments, a vector genome may contain two or more expression cassettes.

The term “translation” in the context of the present invention relates to a process at the ribosome, wherein an mRNA strand controls the assembly of an amino acid sequence to generate a protein or a peptide.

The term “expression” is used herein in its broadest meaning and comprises the production of RNA or of RNA and protein. Expression may be transient or may be stable.

The term “substantial homology” or “substantial similarity,” when referring to a nucleic acid, or fragment thereof, indicates that, when optimally aligned with appropriate nucleotide insertions or deletions with another nucleic acid (or its complementary strand), there is nucleotide sequence identity in at least about 95 to 99% of the aligned sequences. Preferably, the homology is over full-length sequence, or an open reading frame thereof, or another suitable fragment which is at least 15 nucleotides in length. Examples of suitable fragments are described herein.

The terms “sequence identity” “percent sequence identity” or “percent identical” in the context of nucleic acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g., of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length and may be up to about 700 amino acids. Examples of suitable fragments are described herein.

By the term “highly conserved” is meant at least 80% identity, preferably at least 90% identity, and more preferably, over 97% identity. Identity is readily determined by one of skill in the art by resort to algorithms and computer programs known by those of skill in the art.

Generally, when referring to “identity”, “homology”, or “similarity” between two different adeno-associated viruses, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Examples of such programs include, “Clustal Omega”, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference. Multiple sequence alignment programs are also available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

As described above, the term “about” when used to modify a numerical value means a variation of ±10%, unless otherwise specified.

As used throughout this specification and the claims, the terms “comprise” and “contain” and its variants including, “comprises”, “comprising”, “contains” and “containing”, among other variants, is inclusive of other components, elements, integers, steps and the like. The term “consists of” or “consisting of” are exclusive of other components, elements, integers, steps and the like.

It is to be noted that the term “a” or “an”, refers to one or more, for example, “an enhancer”, is understood to represent one or more enhancer(s). As such, the terms “a” (or “an”), “one or more,” and “at least one” is used interchangeably herein.

With regard to the description of these inventions, it is intended that each of the compositions herein described, is useful, in another embodiment, in the methods of the invention. In addition, it is also intended that each of the compositions herein described as useful in the methods, is, in another embodiment, itself an embodiment of the invention.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

### EXAMPLES

The following Examples are provided to illustrate various embodiments of the present invention. The Examples are not intended to limit the invention in any way.

### Example 1: AAV9 Capsid Variants

The gold standard vector for intravenous AAV gene therapy is AAV9. Our group was the first to describe the primary cellular receptor for AAV9, the cell surface glycan galactose. We were further able to identify the amino acid galactose binding pocket on the AAV9 capsid surface, which includes residues D271, N272, Y446, N470 and W503 of the AAV9 VP1 protein. Alanine substitution of any of these residues largely abolished in vitro transduction, or entry and expression of the vector transgene in target cells. We have shown that in vitro transduction deficiency translates to marked in vivo detargeting from the murine liver. Our findings demonstrate that both liver detargeting and peripheral transduction are dependent upon factors within the combinatorial space of the AAV9 galactose binding site. Herein, we describe the identification of novel AAV vectors with improved transduction of target tissues.

### First Round: Directed Evolution and the Galactose Binding Pocket

Directed evolution of AAV vectors involved generation of a vector library. The vector library was produced from a plasmid library containing an array of AAV VP1 variants flanked by AAV ITRs. Using this plasmid library for vector production, we generated a pool of unique AAV vectors, each carrying its unique VP1 coding sequence as the deliverable transgene. This vector was then used for transduction experiments; any cell that was transduced by a library vector expressed the variant's unique VP1 protein. This expression was captured via Next Generation Sequencing (NGS) after RNA extraction from compartments of interest. In vivo injection of one of these libraries thus generated a dataset rich in organ specific transduction patterns as they relate to variation in the vector library. Vector variants with desirable properties were identified from these datasets.

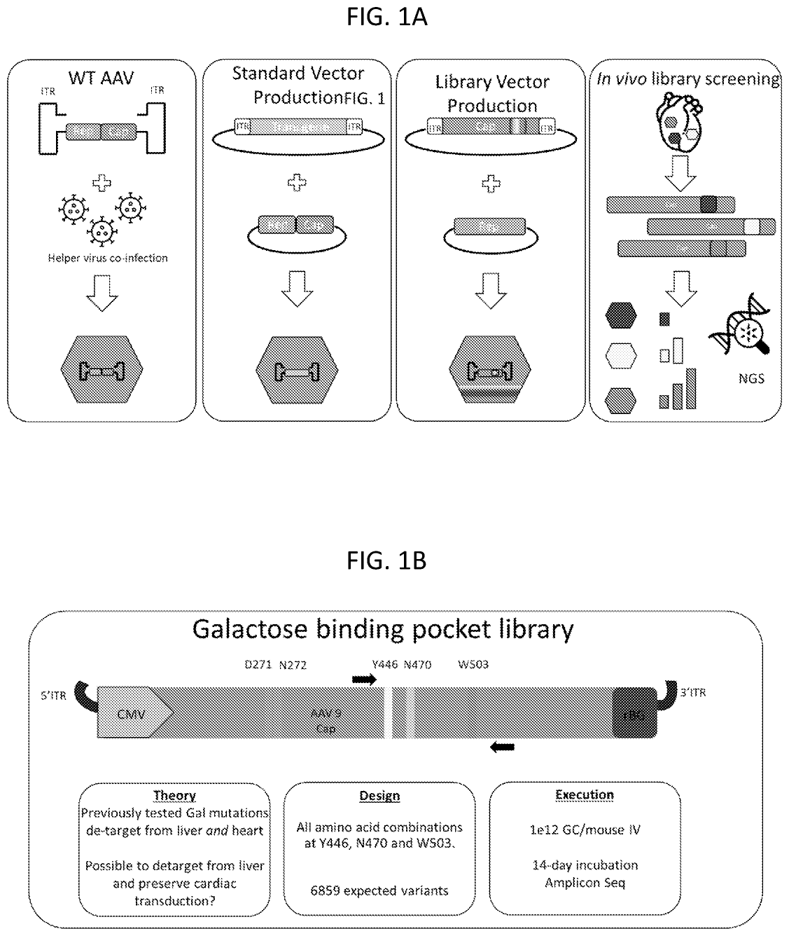

The directed evolution library approach was applied to an analysis of the galactose binding pocket of AAV9. We created an AAV vector library containing variation at three amino acids of the binding pocket, positions 446, 470, and 503 (FIG. 1A and FIG. 1B). Every combination of amino acid substitution at each position was included in the library, resulting in a library containing ˜7000 variants. One of these variants was the WT AAV9 vector, which served as an internal control. This library was then screened in C57Bl/6J mice (n=4, 1e12 GC/mouse, IV injections, Day 14 necropsy), and vector expression was analyzed in liver, heart, muscle, brain, spleen, and kidney tissue via NGS. Sequencing was also performed on the input production plasmid library and the resulting vector library to allow for the derivation of further performance metrics.

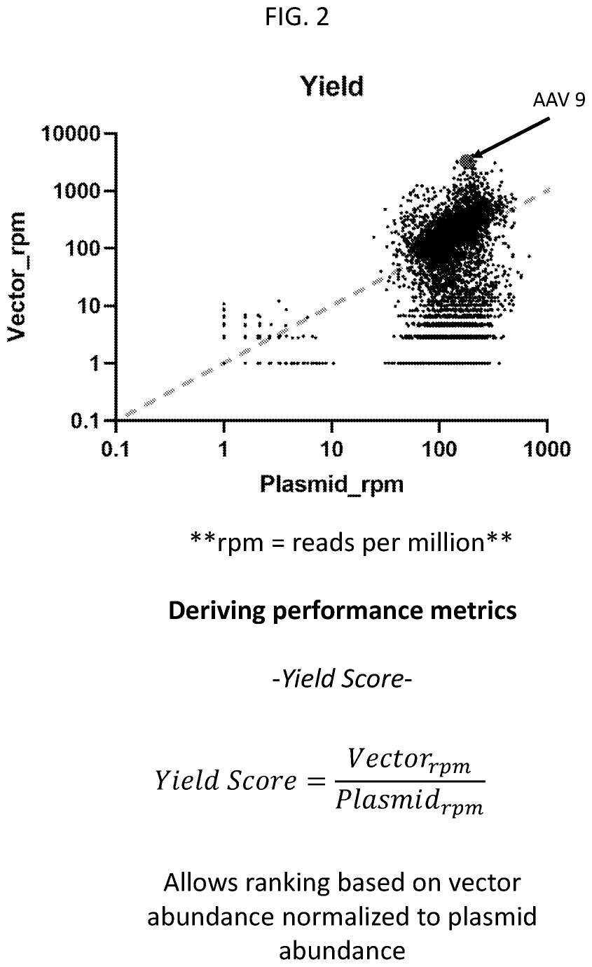

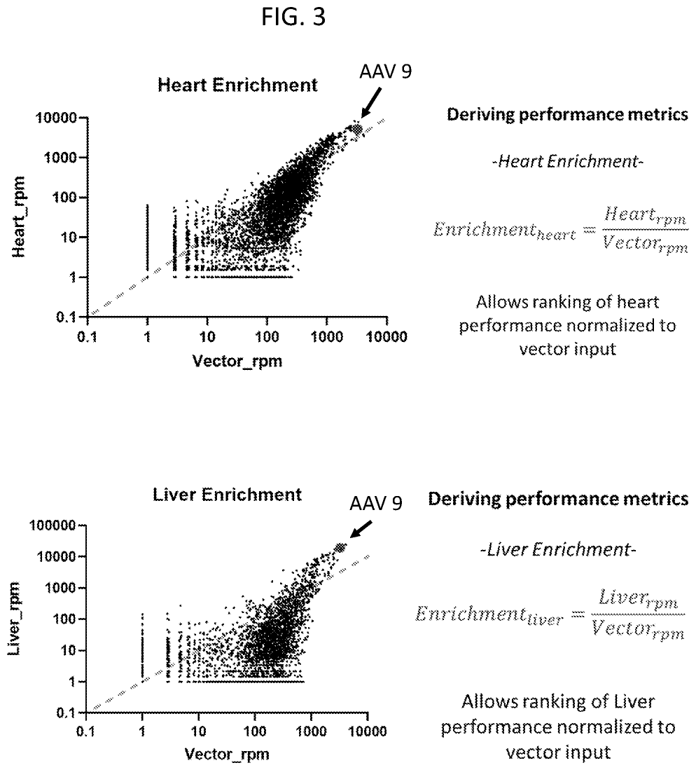

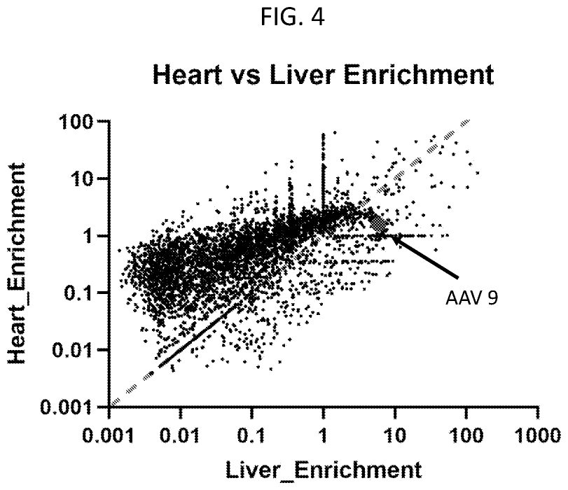

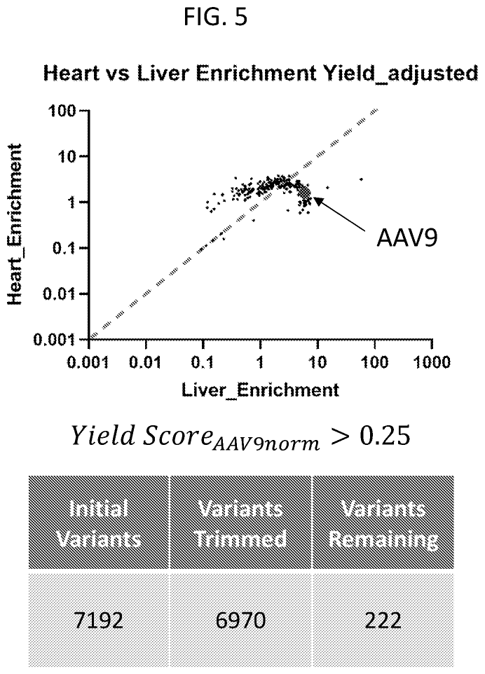

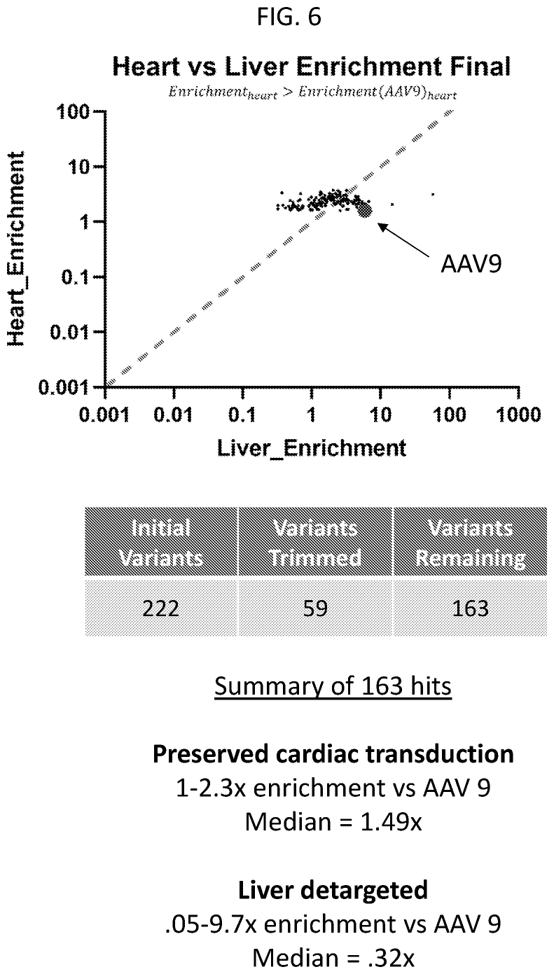

From the results dataset, we determined the yield, liver enrichment, and cardiac enrichment of each vector (FIG. 2, FIG. 3, and FIG. 4). Values were normalized to the AAV9 control to create a standard against which to filter the data and choose variants of interest. The first and most effective filter we applied to these results was a yield filter. For each variant, a yield score was determined by dividing the number of NGS reads in the plasmid library by the number of reads in the vector library. This proportion was then normalized to AAV9, and only vectors with an AAV9 adjusted yield score >0.25 (Yields greater than 25% AAV9) were considered for further analysis (FIG. 5). This filter removed potentially low-yield variants, whose NGS reads and thus performance metrics could be more susceptible to noise in the sequencing data. The yield filter removed most variants from the library, trimming it from ˜7000 variants to ˜200. The next performance metrics used to evaluate the variants were heart and liver enrichment. These were calculated by dividing the NGS reads in a particular tissue by the reads from the vector library. By again normalizing these values to those of AAV9, we were able to screen out variants that performed more poorly than AAV9 in either the heart or the liver. We were left with 163 unique vector variants after applying the heart and liver filters (FIG. 6). All of these variants had appreciable yields, and cardiac or liver enrichment scores above those of AAV9.

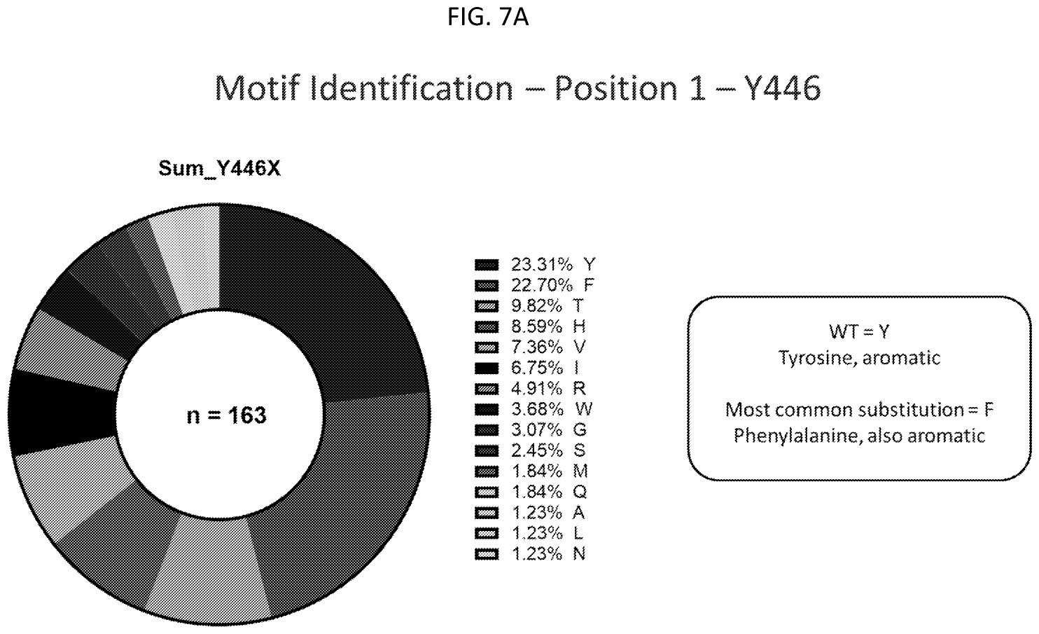

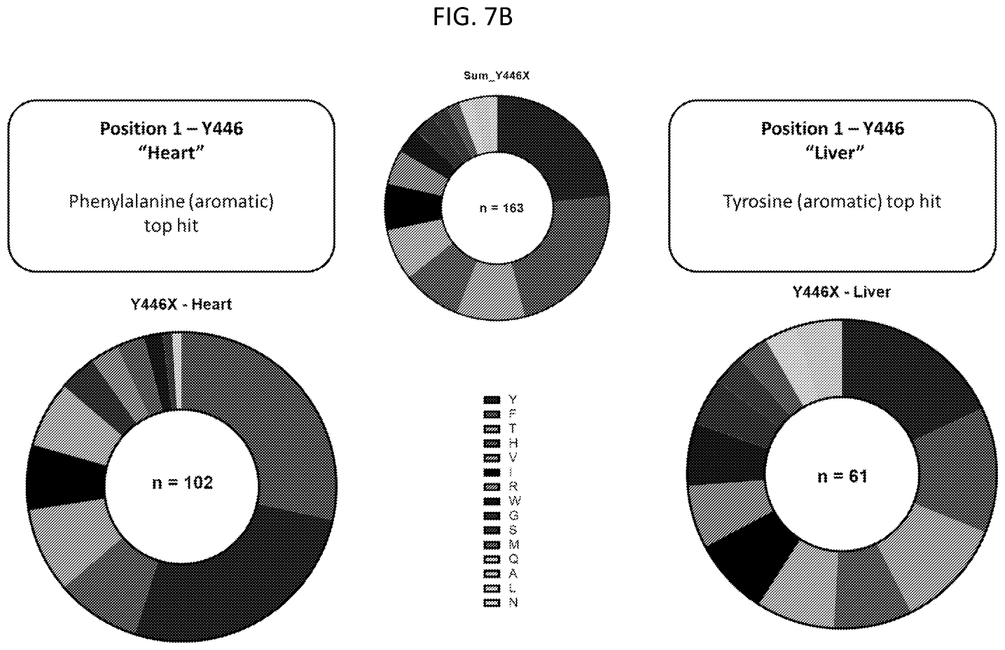

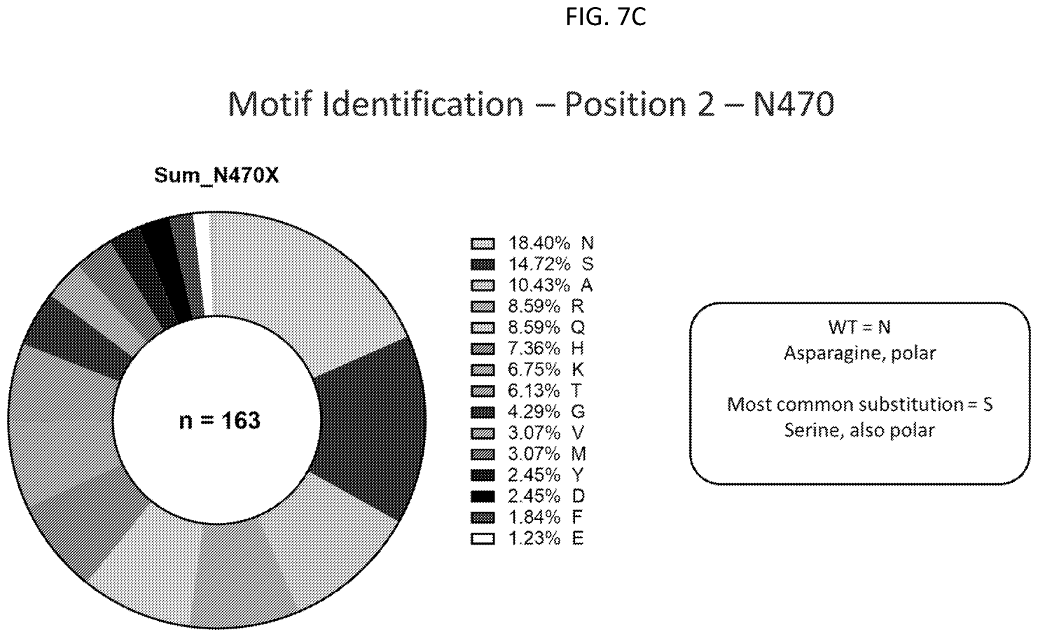

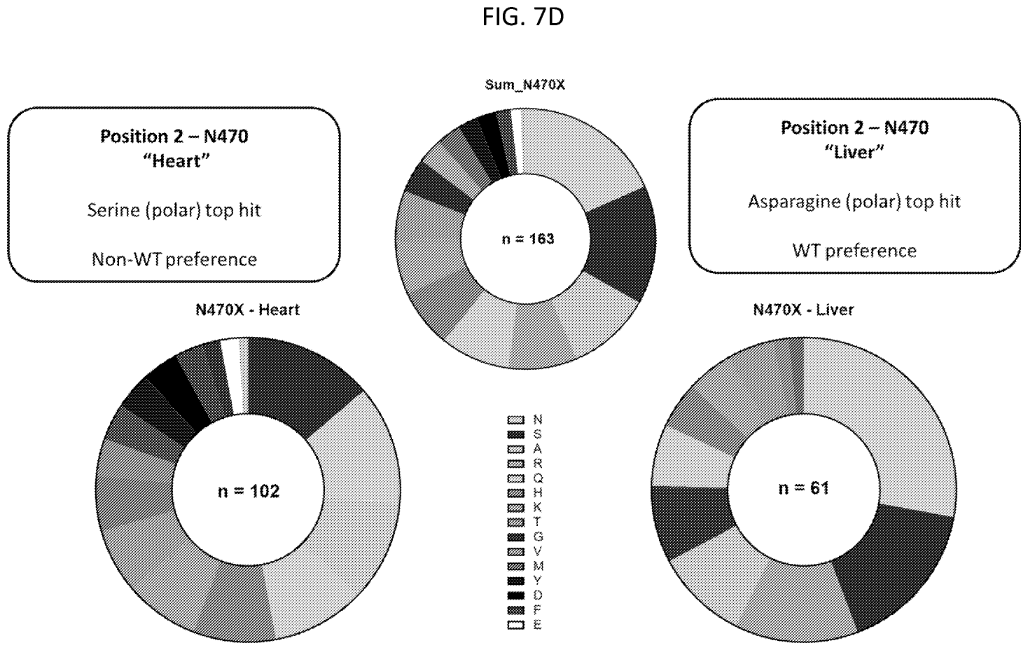

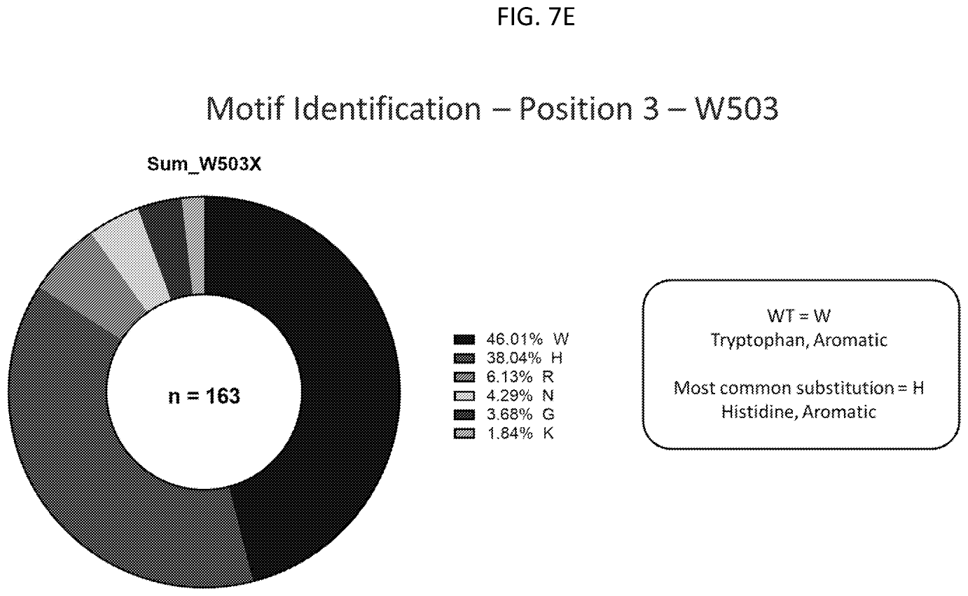

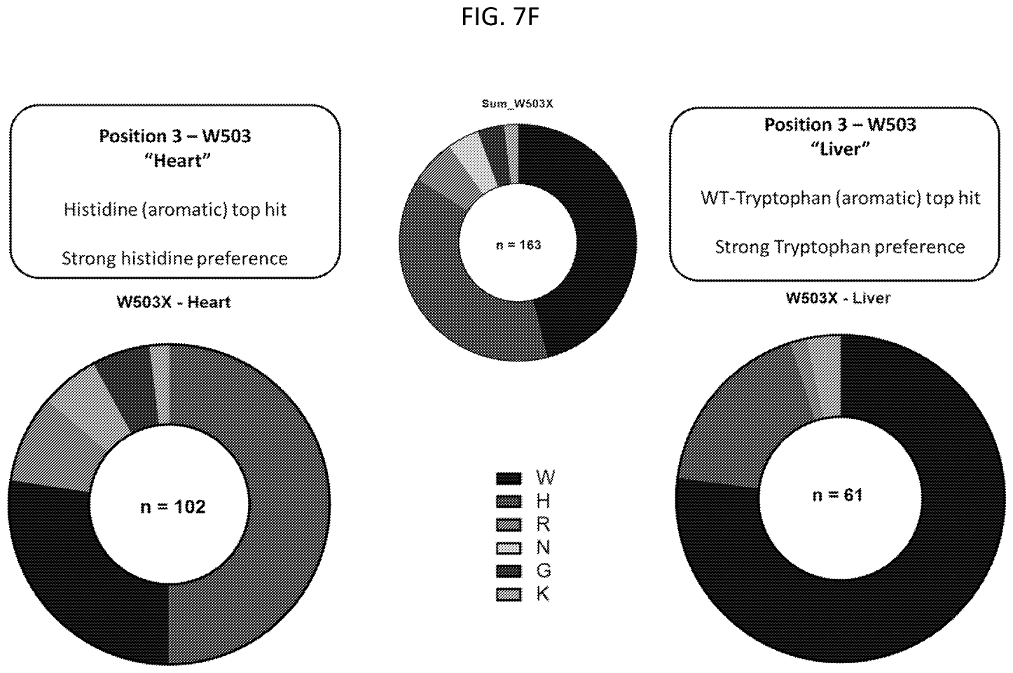

Additional analysis of the vector variants led to the identification of amino acid changes that contribute to altered capsid properties. First, it appeared that position 503 was far more constricted in amino acid composition than the other two positions. While positions 446 and 470 each had 15 amino acids represented in varying proportions, position 503 only had only six. Analysis of these variants in heart and liver indicated a trend wherein variants poorly enriched in the liver had higher cardiac transduction. Positional analysis of these variants indicated a preference for alternative aromatic amino acids, most strikingly at position W503 (FIG. 7A-FIG. 7F). Our findings suggest that that galactose binding affinity correlates positively with liver transduction in vivo, while non-tryptophan aromatics will be favored in vectors that detarget the liver but maintain peripheral transduction.

### Second Round: Directed Evolution and the Galactose Binding Pocket

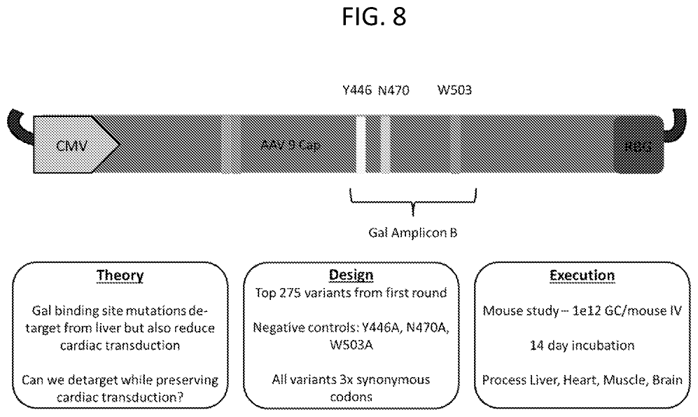

To evaluate whether modifications that reduce galactose binding results in liver detargeting and improved targeting of peripheral tissues including the heart, we generated a second-round library from the top hits of the first-round library (FIG. 8). This new library contained all 163 top performing variants from the first-round library, in addition to both a positive control (AAV9) and negative controls (Y446A, N470A and W503A). While in the first-round library each variant was only coded for by a single DNA sequence, the second round leveraged alternative codon combinations to contain three unique coding sequences for each variant. This extra layer of internal redundancy increased the robustness of the sequencing results. This library was screened in both mouse and NHP and analyzed via Amplicon Seq.

An experimented similar to that described above was performed. The second library (˜200 variants) was injected IV into both C57Bl/6J and BALB-C mice (n=4/group). Mice were sacrificed at D14 post injection, and numerous tissues were harvested for RNA extraction and subsequent NGS analysis. Analysis of this dataset yielded a number useful datapoints. First, it allowed for the ranking of variant enrichment in both the liver and peripheral tissues. Second, the dataset was analyzed to determine higher order relationships between amino acid combinations (not just single amino acid substitutions) as they relate to in vivo transduction patterns. Furthermore, the degree of similarity between the two different strains of mice provided insight into how well galactose binding translates between different species. Top hits from this round were selected for testing on their own in vivo (see Example 2).

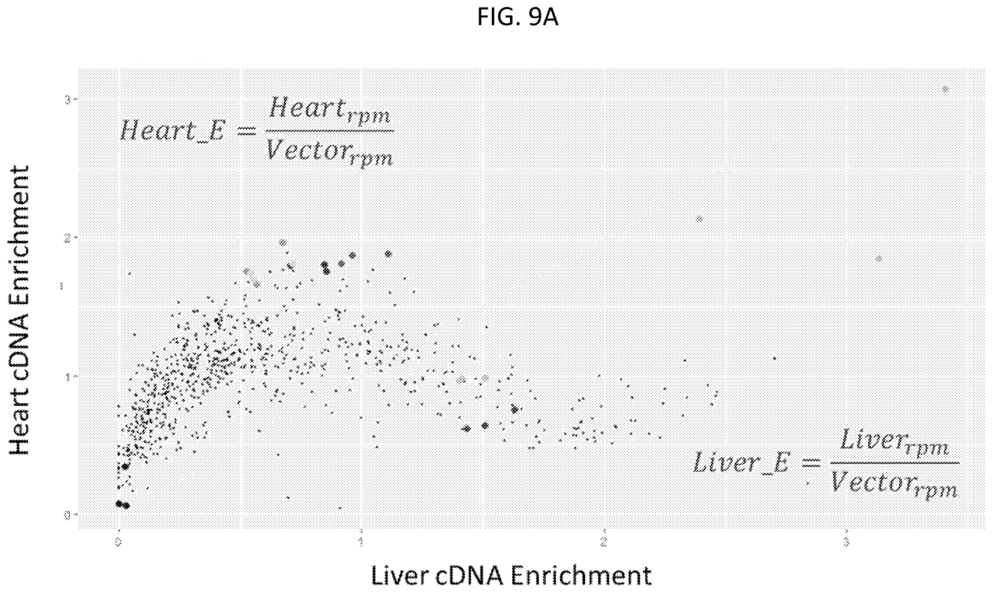

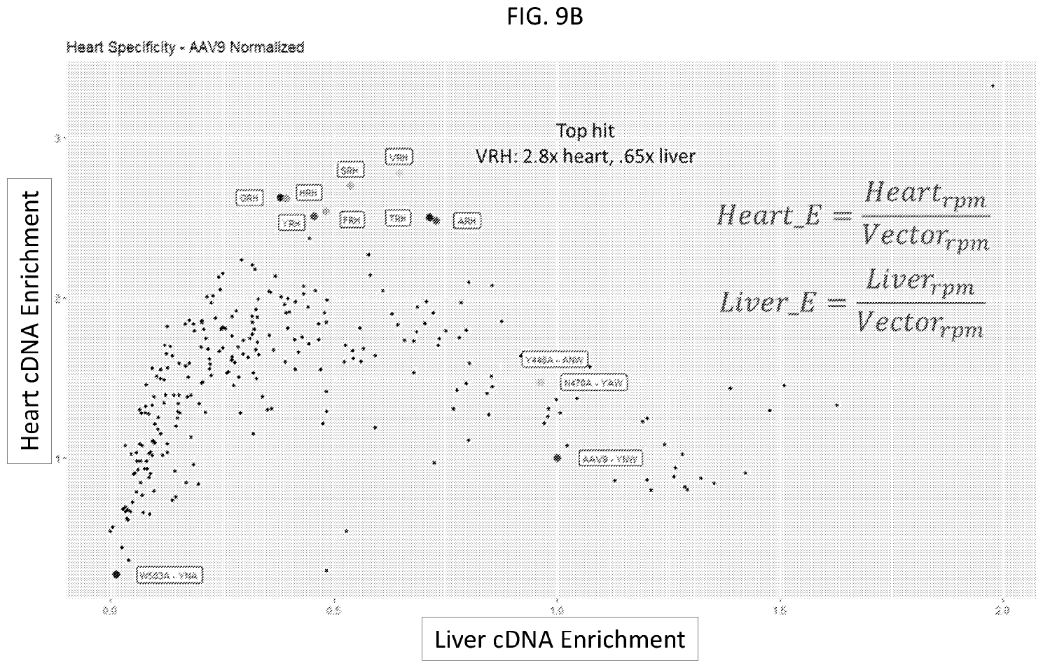

As shown in FIG. 9A and FIG. 9B, a collection of variants that detargeted liver and had enhanced heart transduction were identified. The variants include an XRH motif (i.e., any amino acid (X) at position 446, R at position 470, and H at position 503). The top hit, a VRH variant, had about a 2.7-fold increase heart transduction and a 0.4× reduction liver transduction.

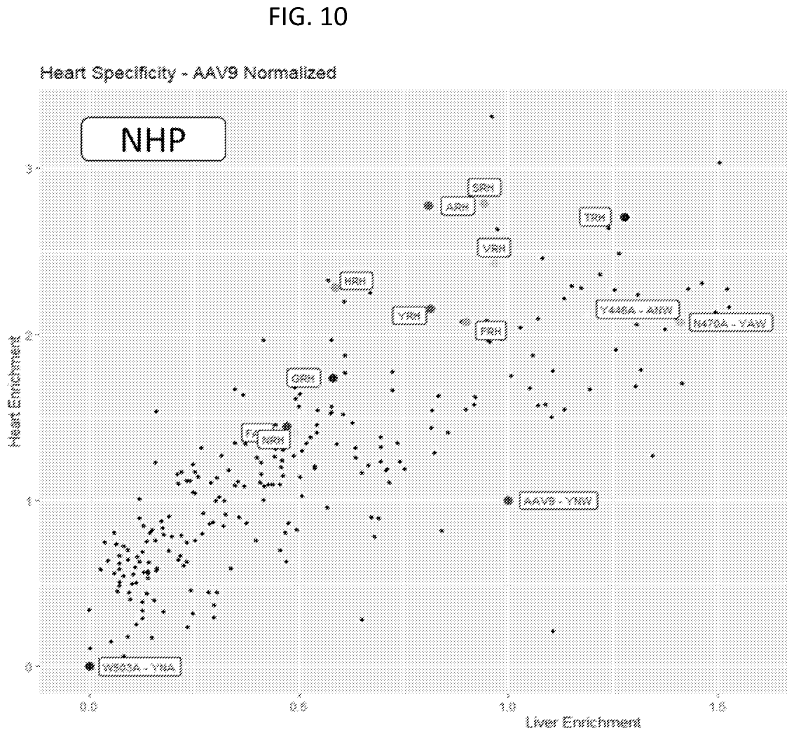

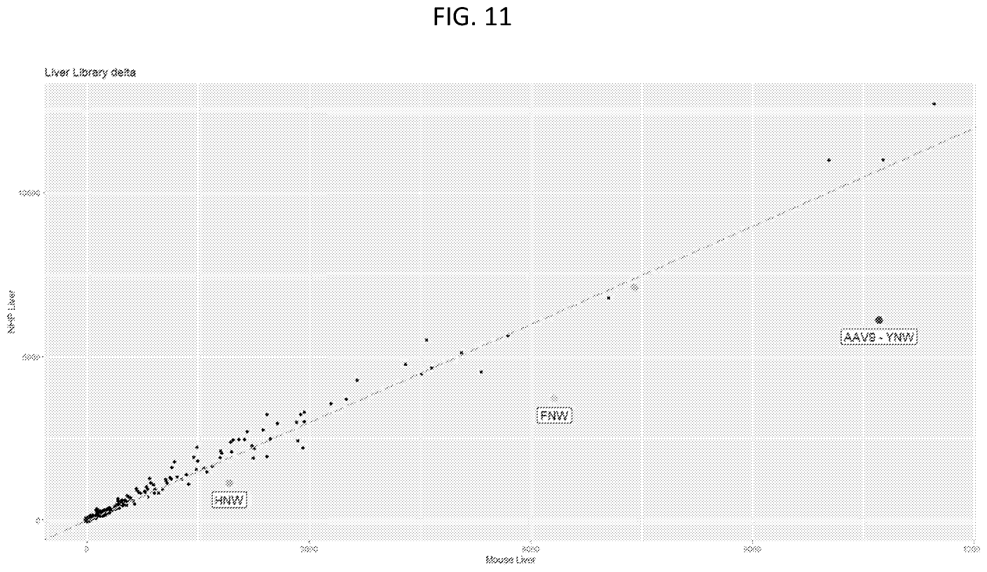

While the cardiac enrichment trend remained in NHP, the liver detargeting trend was no consistent (FIG. 10). This was not due to a change in any of the novel variants, but instead due to a difference in AAV9 liver performance between mouse and NHP. Most variants performed similarly in mouse and NHP liver, whereas AAV9 and its close cousins had a lower RPM in NHP liver than they did in mouse liver (FIG. 11). This suggested a dependency on galactose binding and prompted further testing of library variants in vitro.

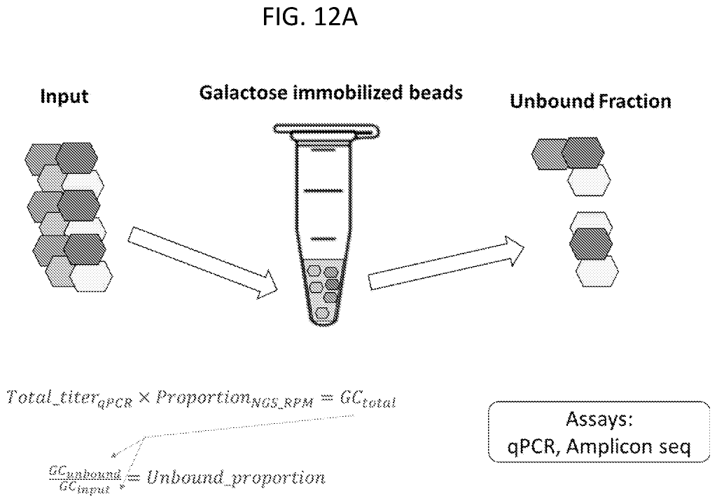

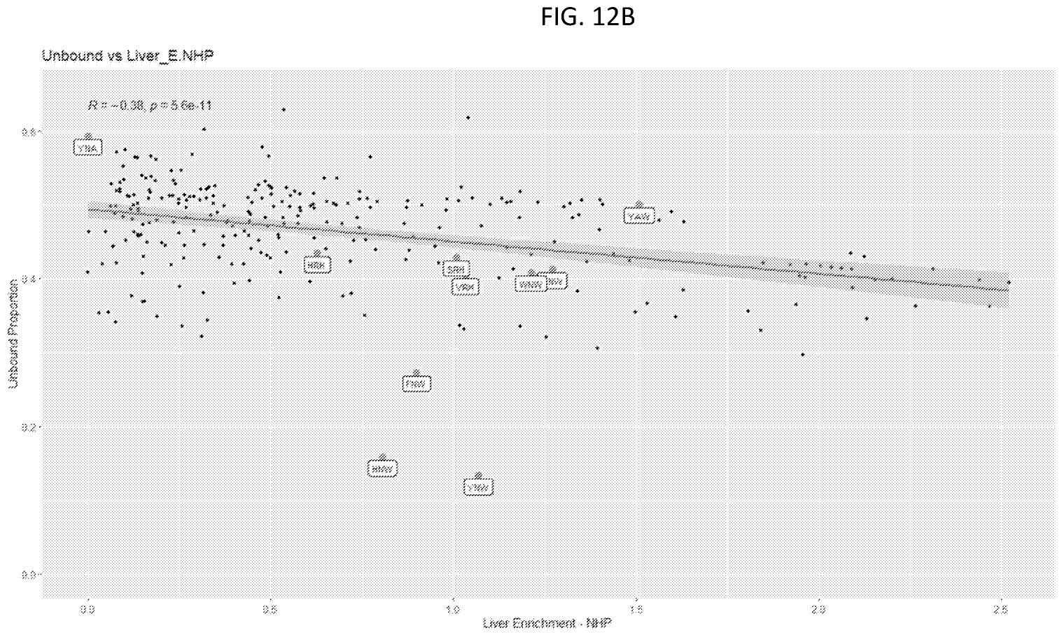

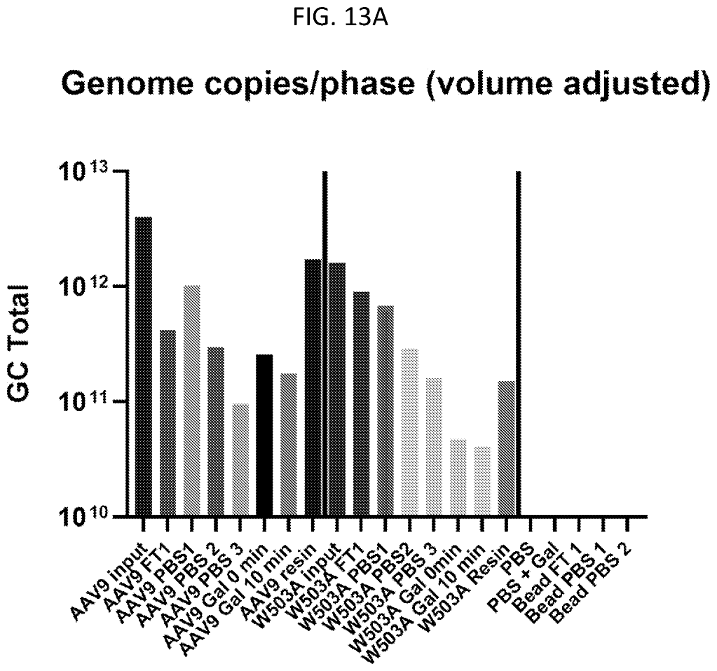

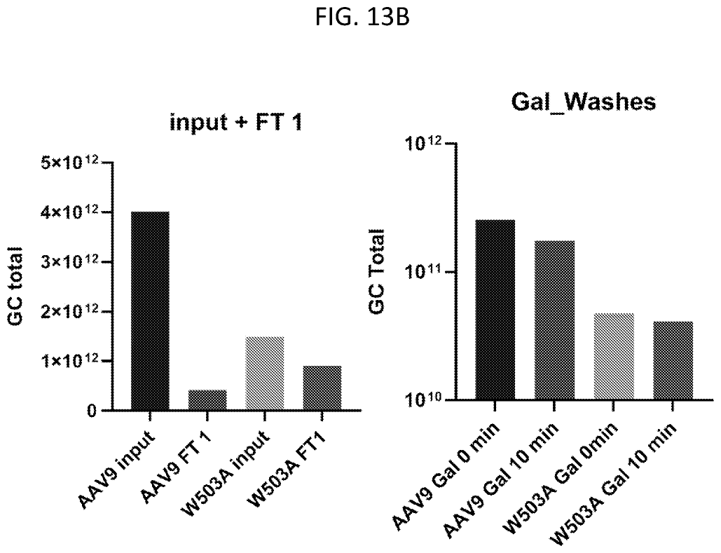

To further test galactose binding of the individual variants, we examined the binding of the second-round library to agarose beads with immobilized galactose. The vector library was allowed to bind to galactose beads for an hour (in a tube turner), at which point the mix was spun down and the supernatant removed (FIG. 12A). qPCR and Amplicon-seq of both the input and the supernatant enabled calculation of the proportion of input vector that was detected in the unbound fraction. The vector having the W503A mutation was abundant in flowthrough and washes, and analysis of the unbound proportion indicated that most of the tested variants have very low binding to bead-bound galactose. The exceptions were AAV9 and its two close cousins, FNW and HNW (FIG. 12B). FIG. 13A and FIG. 13B provide results from control studies to validate AAV9 binding to galactose binding in vitro.

Overview of methods to assess galactose binding affinity:
1. Load column w/100 μl bead resin
2. Wash column w/1.2 mL PBS
3. Apply undiluted vector to column—obtain flowthrough (FT1)
4. Wash 3× w/400 μl PBS->PBS 1-3
5. Apply 400 μl 0.1M Galactose, spin immediately->Gal 0 min
6. Apply 400 μl 0.1M Galactose, incubate RT for 10 min->Gal 10 min
7. Resuspend resin in 400 μl PBS->Resin
8. Assay GC in each fraction, adjust for volume

The results identify AAV9 variants with advantages to AAV9 in both cardiac transduction and liver detargeting. Most of these variants have a diminished ability to bind galactose compared to AAV9 and its close cousins. Our findings suggest that these new variants have either intermediate galactose binding or are re-targeted to a different receptor. Furthermore, this work exposed a difference between mouse and NHP liver, likely due to presence of vector-accessible galactose.

For further analysis, the second-round library is also tested in vitro on a range of cell lines in different conditions. Neuraminidase is an enzyme the desialylates terminal glycans, exposing terminal galactose and strongly increasing AAV9's transduction of 293 cells. By transducing 293 cells+/−neuraminidase with the second-round library, we are further able to correlate galactose availability with the transduction patterns of each variant. It is expected that peripherally transducing variants will have higher basal levels of 293 transduction, that will increase to a lesser degree than liver transducing variants after neuraminidase treatment. This experiment can be run in parallel using the CHO lec-2 cell line, which presents desialylate galactose on its surface and has been shown to be susceptible to AAV9 transduction.

### Example 2: Delivery of Individual Vectors to Mice

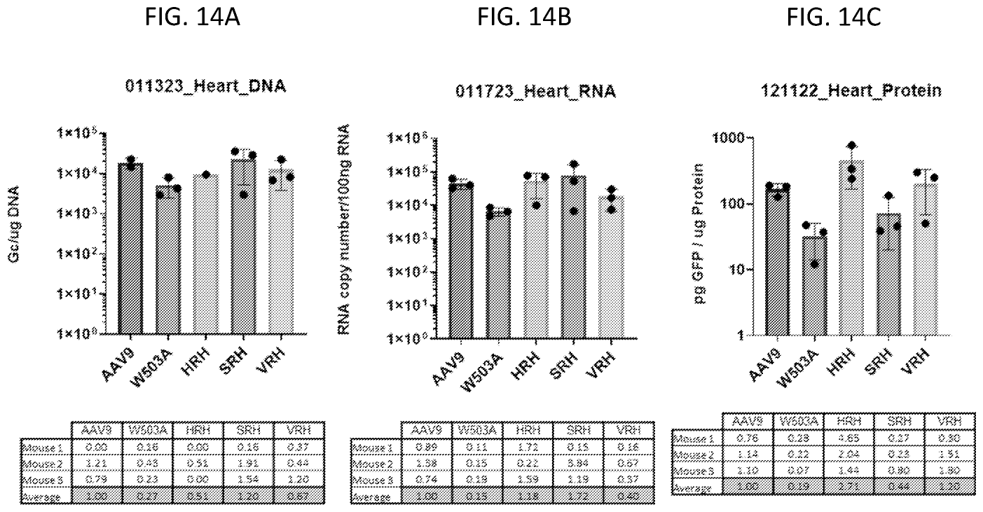

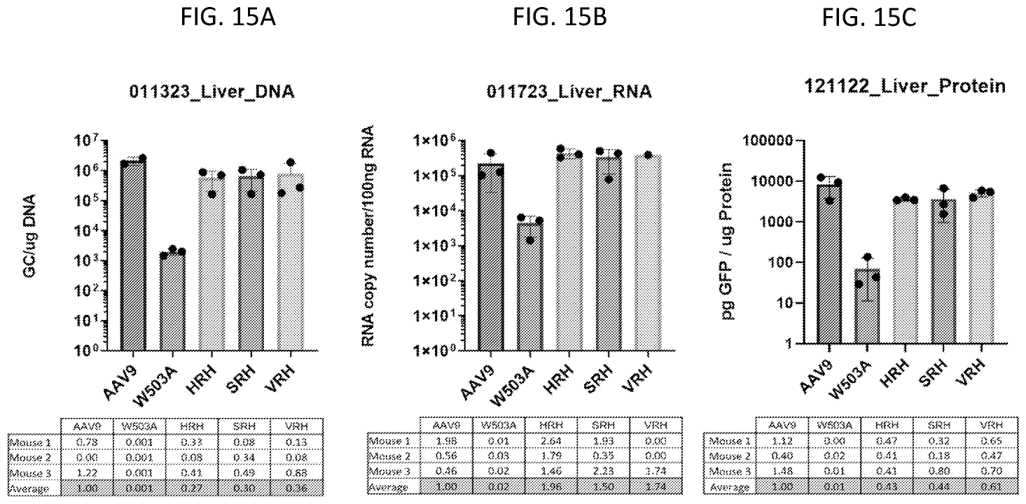

A preliminary study was performed to evaluate transduction and expression of a transgene (GFP) following administration of single vectors having HRH, SRH, or VRH capsid variants (identified as described in Example 1). Each of the capsids was used to package a vector genome having a eGFP transgene operably linked to a CB7 promoter (CB7.CI.eGFP.WPRE.rBG; CI=chimeric intron; rBG=rabbit beta-globin polyA). In addition, control vectors having an AAV9 capsid or an AAV9 capsid with the W503A mutation were generated. Each of the vectors was administered (1×10¹² genome copies (GC)/mouse) to three mice, and necropsies were performed on day 14. Heart and liver tissues were harvested to evaluate transduction (DNA) and RNA and protein expression of the eGFP transgene. FIG. 14A-FIG. 14C and FIG. 15A-FIG. 15C show transduction and expression levels in heart and liver, respectively, including values normalized to transduction and expression observed with AAV9.

All documents cited in this specification are incorporated herein by reference. The priority documents, U.S. Provisional Patent Application No. 63/302,912, filed Jan. 25, 2022, U.S. Provisional Patent Application No. 63/375,005, filed Sep. 8, 2022, and U.S. Provisional Patent Application No. 63/386,572, filed Dec. 8, 2022, are incorporated herein by reference. While the invention has been described with reference to particular embodiments, it will be appreciated that modifications can be made without departing from the spirit of the invention. Such modifications are intended to fall within the scope of the appended claims.

1. A recombinant adeno-associated virus (rAAV) having an AAV clade F capsid comprising a mutant galactose binding site,
wherein the mutant galactose binding site comprises (a) any amino acid residue (X) at position 446 (Y446X), (b) an arginine at position 470 (N470R), and (c) a histidine at position 503 (W503H) when residue positions are determined using the residue numbers of SEQ ID NO: 10 as a reference, and
a vector genome comprising a 5′ inverted terminal repeat (ITR) sequence, a coding sequence for a gene product, regulatory sequences operably linked to the coding sequence that direct expression of the gene product, and a 3′ ITR sequence.

2. The rAAV according to claim 1, wherein the mutant capsid comprises:
(a) H at position 446 (HRH);
(b) S at position 446 (SRH);
(c) V at position 446 (VRH);
(d) G at position 446 (GRH);
(e) F at position 446 (FRH);
(f) T at position 446 (TRH); or
(g) S at position 446 (SRH).

3. The rAAV according to claim 1, wherein the parental AAV clade F capsid is an AAV9, AAVhu68, AAVhu31, AAVhu32, AAVhu95, or AAVhu96 capsid, or a mutant or variant of any of the aforementioned.

4. The rAAV according to claim 1, wherein the mutant clade F capsid further comprises an insertional mutation in the HVR8 locus between position 588 and 589, based on the numbering of the amino acid sequence of SEQ ID NO: 10.

5. The rAAV according to claim 4, wherein the insertional mutation comprises:
(a) the motif N- x- (T/I/V/A)-(K/R) (SEQ ID NO: 19), optionally wherein motif is flanked by two to seven amino acids at its carboxy- and/or amino terminus;
(b) an exogenous targeting peptide having the sequence: optional N-terminal linker- Y-G/A/R/K-Y/H-GNPA-T/R/H-RYFD-V/K (SEQ ID NO: 13)—optional C-terminal linker; inserted between amino acids 588 and 589 of an AAV9 capsid protein, based on the numbering of amino acid sequence: SEQ ID NO: 10; or
(c) a peptide comprising an RGD motif.

6. The rAAV according to claim 5, wherein the capsid further comprises the motif of (a) which is NTVK, optionally selected from:
(SEQ ID NO: 14)
(a) SSNTVKLTSGH;
(SEQ ID NO: 12)
(b) EFSSNTVKLTS;
(SEQ ID NO: 18)
(c) GGVLTNIARGEYMRGG;
(SEQ ID NO: 17)
(d) GGIEINATRAGTNLGG;
(SEQ ID NO: 13)
(e) GGSSNTVKLTSGHGG;
(SEQ ID NO: 16)
(f) IEINATRAGTNL;
or
(SEQ ID NO: 15)
(g) SANFIKPTSY.

7. The rAAV according to claim 5, wherein the capsid further comprises the motif of (b) selected from:
(SEQ ID NO: 28)
(a) Y-G/A/R/K-Y/H-GNPA-T/R/H-RYFD-V/K;
(SEQ ID NO: 20)
(b) YGYGNPATRYFDV;
(SEQ ID NO: 25)
(c) YGYGNPARRYFDV;
(SEQ ID NO: 26)
(d) YGYGNPAHRYFDV;
(SEQ ID NO: 27)
(e) YGYGNPATRYFDK;
(SEQ ID NO: 21)
(f) YAYGNPATRYFDV;
(SEQ ID NO: 22)
(g) YKYGNPATRYFDV;
(SEQ ID NO: 23)
(h) YRYGNPATRYFDV;
or
(SEQ ID NO: 24)
(i) YGHGNPATRYFDV.

8. The rAAV according to claim 1, wherein the regulatory sequences comprise a constitutive promoter.

9. The rAAV according to claim 1, wherein the regulatory sequences comprise a tissue-specific promoter.

10. The rAAV according to claim 9, wherein the tissue-specific promoter is a cardiac promoter.

11. A method for generating an a rAAV comprising a mutant capsid derived from a parental AAV capsid having liver specificity, the method comprising culturing a packaging host cell comprising:
(a) a nucleic acid sequence encoding a mutant AAV Clade F capsid operably linked to regulatory control sequences that direct its expression in the packaging host cell, wherein the encoded capsid protein comprises (i) any amino acid residue (X) at position 446 (Y446X), (ii) an arginine at position 470 (N470R), and (iii) a histidine at position 503 (W503H), where the amino acid residue positions are determined using the residue numbers of SEQ ID NO: 10 as a reference,
(b) a nucleic acid molecule comprising a vector genome comprising a 5′ inverted terminal repeat (ITR) sequence, a coding sequence for a gene product, regulatory sequences operably linked to the coding sequence that direct expression of the gene product, and a 3′ ITR sequence; and
(c) helper functions necessary for packaging the vector genome of (b) into the mutant Clade F capsid.

12. The method according to claim 11, wherein the parental AAV capsid is an AAV9, AAVhu68, AAVhu31, AAVhu32, AAVhu95, or AAVhu96 capsid, or a mutant or variant of any of the aforementioned.

13. An rAAV produced according to the method of claim 11.

14. A method for reducing liver toxicity associated with delivery of an rAAV vector, the method comprising delivering to a subject the rAAV according to claim 1.

15. A method for improved delivery of a gene product to cardiac cells or tissue, the method comprising administering to a subject the rAAV according to claim 1.

16. The method according to claim 14, wherein the coding sequence comprises a LaminA gene, Kv11.1 voltage-gated potassium channel (ERG) gene, a tafazzin (TAZ gene, potassium voltage-gated channel subfamily Q member 1 (KCNQ1) gene, or a cardiac myosin binding protein C (MYBPC3) gene.

17. A nucleic acid comprising a sequence encoding a mutant AAV Clade F VP1 protein having a mutant galactose binding pocket which comprises (a) any amino acid residue (X) at position 446 (Y446X), (b) arginine at position 470 (N470R), and (c) histidine at position 503 (W503H), where the amino acid residue positions are determined using the residue numbers of SEQ ID NO: 10 as a reference, the VP1 protein coding sequence being operably linked to expression control sequences which direct its expression in a packaging host cell.

18. The nucleic acid according to claim 17, wherein the nucleic acid is a plasmid.

19. A host cell comprising the nucleic acid according to claim 17.


SEQUENCE LISTING (28 sequences)

SEQ ID NO: 1 - protein, 736 aa, synthetic construct
  REGION 1..736: note = synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYVLSKTINGSGQNQQTLKFSVAGPSRMAVQGRNYIPGPSYRQQRVSTTVTQNNNSEFAHPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 736 aa, synthetic construct
  REGION 1..736: note = synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYSLSKTINGSGQNQQTLKFSVAGPSRMAVQGRNYIPGPSYRQQRVSTTVTQNNNSEFAHPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - protein, 736 aa, synthetic construct
  REGION 1..736: note = synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYHLSKTINGSGQNQQTLKFSVAGPSRMAVQGRNYIPGPSYRQQRVSTTVTQNNNSEFAHPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 4 - protein, 736 aa, synthetic construct
  REGION 1..736: note = synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYGLSKTINGSGQNQQTLKFSVAGPSRMAVQGRNYIPGPSYRQQRVSTTVTQNNNSEFAHPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 5 - protein, 736 aa, synthetic construct
  REGION 1..736: note = synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYFLSKTINGSGQNQQTLKFSVAGPSRMAVQGRNYIPGPSYRQQRVSTTVTQNNNSEFAHPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 6 - protein, 736 aa, synthetic construct
  REGION 1..736: note = synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSRMAVQGRNYIPGPSYRQQRVSTTVTQNNNSEFAHPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 7 - protein, 736 aa, synthetic construct
  REGION 1..736: note = synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYTLSKTINGSGQNQQTLKFSVAGPSRMAVQGRNYIPGPSYRQQRVSTTVTQNNNSEFAHPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 8 - protein, 736 aa, synthetic construct
  REGION 1..736: note = synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYALSKTINGSGQNQQTLKFSVAGPSRMAVQGRNYIPGPSYRQQRVSTTVTQNNNSEFAHPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 9 - protein, 736 aa, synthetic construct
  VARIANT 446
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYXLSKTINGSGQNQQTLKFSVAGPSRMAVQGRNYIPGPSYRQQRVSTTVTQNNNSEFAHPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - protein, 736 aa, Adeno-associated virus hu68
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 12 - protein, 11 aa, synthetic construct
  REGION 1..11: note = EFS peptide sequence
EFSSNTVKLTS

SEQ ID NO: 13 - protein, 15 aa, synthetic construct
  REGION 1..15: note = SSN-L peptide sequence
GGSSNTVKLTSGHGG

SEQ ID NO: 14 - protein, 11 aa, synthetic construct
  REGION 1..11: note = SSN peptide sequence
SSNTVKLTSGH

SEQ ID NO: 15 - protein, 10 aa, synthetic construct
  REGION 1..10: note = SAN peptide sequence
SANFIKPTSY

SEQ ID NO: 16 - protein, 12 aa, synthetic construct
  REGION 1..12: note = IEI peptide sequence
IEINATRAGTNL

SEQ ID NO: 17 - protein, 16 aa, synthetic construct
  REGION 1..16: note = IEI-L peptide sequence
GGIEINATRAGTNLGG

SEQ ID NO: 18 - protein, 16 aa, synthetic construct
  REGION 1..16: note = VLT-L peptide sequence
GGVLTNIARGEYMRGG

SEQ ID NO: 19 - skipped

SEQ ID NO: 20 - protein, 13 aa, synthetic construct
  REGION 1..13: note = YGY peptide sequence
YGYGNPATRYFDV

SEQ ID NO: 21 - protein, 13 aa, synthetic construct
  REGION 1..13: note = YGY2A peptide sequence
YAYGNPATRYFDV

SEQ ID NO: 22 - protein, 13 aa, synthetic construct
  REGION 1..13: note = YGY2K peptide sequence
YKYGNPATRYFDV

SEQ ID NO: 23 - protein, 13 aa, synthetic construct
  REGION 1..13: note = YGY2R peptide sequence
YRYGNPATRYFDV

SEQ ID NO: 24 - protein, 13 aa, synthetic construct
  REGION 1..13: note = YGY3H peptide sequence
YGHGNPATRYFDV

SEQ ID NO: 25 - protein, 13 aa, synthetic construct
  REGION 1..13: note = YGY8R peptide sequence
YGYGNPARRYFDV

SEQ ID NO: 26 - protein, 13 aa, synthetic construct
  REGION 1..13: note = YGY8H peptide sequence
YGYGNPAHRYFDV

SEQ ID NO: 27 - protein, 13 aa, synthetic construct
  REGION 1..13: note = YGY13K peptide sequence
YGYGNPATRYFDK

SEQ ID NO: 28 - protein, 13 aa, synthetic construct
  REGION 1..13: note = Y-G/A/R/K-Y/H-GNPA-T/R/H-RYFD-V/K motif
  VARIANT 2: note = Xaa is selected from Glycine (G), Alanine (A), Arginine (R), or Lysine (K)
  VARIANT 3: note = Xaa is selected from Tyrosine (Y), or Histidine (H)
  VARIANT 8: note = Xaa is selected from Threonine (T), Arginine (R), or Histidine (H)
  VARIANT 13: note = Xaa is selected from Valine (V), or Lysine (K)
YXXGNPAXRYFDX